SEQUENCE LISTING (2 sequences)

SEQ ID NO: 1 - DNA, 20 bp, Artificial Sequence
  primer sequence.
atgaaaatcctcgtggcggt

SEQ ID NO: 2 - DNA, 19 bp, Artificial Sequence
  primer sequence
cagcatcccgttgcccatt

(19) United States
(12) Patent Application Publication
(10) Pub. No.: US 2009/0312255 A1
(43) Pub. Date: Dec. 17, 2009

(54) STIMULATION OF TRPV1+ SENSORY NEURONS TO CONTROL BETA-CELL STRESS AND ISLET INFLAMMATION IN DIABETES

(75) Inventors: Hans-Michael DOSCH, Toronto (CA); Lan Tang, Toronto (CA); Yin Chan, Toronto (CA); Michael Salter, Etobicoke (CA)

Correspondence Address: Vedder Price, PC, 875 15th Street, NW, Suite 725, Washington, DC, 20005, US

(73) Assignee: The Hospital for Sick Children, Toronto (CA)

(21) Appl. No.: 12/478,898

(22) Filed: Jun. 5, 2009

Related U.S. Application Data
(63) Continuation-in-part of application No. 12/394,261, filed on Feb. 27, 2009.
Application No. 12/394,261 is a division of application No. 11/638,830, filed on Dec. 14, 2006, now Pat. No. 7,544,365.

(30) Foreign Application Priority Data
Dec. 13, 2007 (CA) PCT/CA07/02254

(51) Int. Cl. A61K 31/165; A61P 3/10; A61K 38/17

(52) U.S. Cl. 514/12; 514/627

(57) ABSTRACT

The present invention provides a method of altering the function of TRPV1+ sensory afferent neurons in the pancreas as a way of treating, managing, alleviating, etc., the symptoms and/or underlying causes of diabetes or abnormal glucose metabolism by increasing the release of neuropeptides, such as substance P (sP) or other tachykinin peptide, in the pancreas. This may be achieved by injecting a TRPV1 agonist, such as a capsaicinoid compound or capsaicin analog, or a neuropeptide, such as sP or other tachykinin peptide, directly into the pancreas, or alternatively, by stimulating one or more intercostal and/or subcostal nerves of spinal nerves derived from one or more thoracic segments T8 through T12 by chemical, electrical, surgical, mechanical, etc., methods.

### CROSS-REFERENCE TO RELATED APPLICATIONS

This application claims benefit of priority to U.S. patent application Ser. No. 12/394,261, filed Feb. 27, 2009, which is a divisional application claiming the benefit of priority to U.S. patent application Ser. No. 11/638,830, filed Dec. 14, 2006. The contents and disclosures of these applications are hereby incorporated by reference in their entirety.

### FIELD OF THE INVENTION

The present invention relates to methods of treating individuals having or at risk of developing diabetes or other conditions characterized by abnormal glucose regulation or metabolism.

### BACKGROUND

Diabetes has traditionally been divided into two main classifications including Type-1 diabetes (T1D) and Type-2 diabetes (T2D). T1D is an autoimmune disease, where the insulin-producing β-cells become autoimmune targets of a permissive immune system and are destroyed over the usually prolonged period of clinically silent ‘pre-diabetes’ that progresses slowly (over about 10 years in humans) towards overt insulin deficiency. Infiltrating autoreactive T-cells penetrate the layer of peri-islet Schwann cells (pSC) to gain access to the endocrine β-cell mass. T1D was formerly referred to as insulin-dependent diabetes mellitus (IDDM) or “juvenile diabetes.” However, these terms are no longer preferred because insulin therapy is no longer unique to T1D, and T1D may occur at any age with a presentation of insulin resistance. T1D has been shown to be influenced by and associated with particular genetic backgrounds as well as environmental factors, such as infection, which may increase penetrance of the disease. T2D is a metabolic disorder that generally results from insulin resistance in peripheral tissues and is primarily observed in adults, but with rising incidence in children and adolescents. Obesity is a common risk factor associated with T2D. T2D was formerly referred to as non-insulin-dependent diabetes mellitus. However, this term is no longer preferred since current T2D therapy is more commonly using insulin.

Hybrid or intermediate forms of diabetes having a combination of characteristics traditionally associated with either T1D or T2D have also been identified, suggesting more of a continuum between insulin deficiency and insulin resistance in diabetes, rather than discrete classifications of the disease. Indeed, both T1D and T2D share fundamental similarities. See, e.g. Alberti, K. G. et al., “Definition, diagnosis, and classification of diabetes mellitus and its complications. Part 1: diagnosis and classification of diabetes mellitus provisional report of a WHO consultation,” Diabet Med 15:539-553 (1998); and Lernmark, A., “Type 1 diabetes,” Clin Chem 45:1331-1338 (1999), the entire contents and disclosures of which are hereby incorporated by reference. For example, some patients diagnosed with T2D in fact have autoimmune manifestations and may have been misdiagnosed. This patient group is designated as Latent Autoimmune Diabetes of the Adult (LADA), a late-onset T1D cohort with a less aggressive course than typical T1D. See, e.g. Chiu, H. K. et al., “Equivalent insulin resistance in latent autoimmune diabetes in adults (LADA) and type 2 diabetic patients,” Diabetes Res Clin Pract 77:237-244 (2007); Goel, A. et al., “T-cell responses to islet antigens improves detection of autoimmune diabetes and identifies patients with more severe beta-cell lesions in phenotypic type 2 diabetes,” Diabetes 56(8):2110 (2007); and Pozilli, P. et al., “Autoimmune Diabetes Not Requiring Insulin at Diagnosis (Latent Autoimmune Diabetes of the Adult): Definition, characterization, and potential prevention,” Diabetes Care 24:1460-1467 (2001), the entire contents and disclosures of which are hereby incorporated by reference. Conversely, with the increase in childhood obesity, there is an increase in T2D in young children. These children present with insulin resistance, the core symptom of T2D, but often have clear signs of autoimmunity typical of T1D. See, e.g. Donath, M. Y. et al., “Type 1, type 1.5, and type 2 diabetes: NOD the diabetes we thought it was,” PNAS USA 103:12217-12218 (2006), the entire contents and disclosure of which is hereby incorporated by reference. Clinicians consider these children “Type-1.5” diabetics. More recently, Type-3 Diabetes (T3D) has been identified in association with Alzheimer's Disease. See, e.g., Messier, C. et al., “The role of insulin, insulin growth factor, and insulin-degrading enzyme in brain aging and Alzheimer's disease,” Neural Plast 12:311-328 (2005), the entire contents and disclosure of which is hereby incorporated by reference.

Both T1D and T2D may be characterized by insulin resistance as well as a progressive lack of sufficient insulin reserves. In general, autoimmunity depletes β-cell mass in T1D, whereas β-cell stress in T2D limits the large amounts of insulin required in the face of progressively increasing insulin resistance. It is becoming increasingly clear that after decades of considering T1D and T2D as different disorders, these classifications may now be viewed as different extremes of similar underlying conditions superimposed on differing genetic backgrounds and environmental factors. Due to their shared pathophysiological properties, novel compositions and treatment methods may have therapeutic potential in treating one or more different types of diabetes.

Insulin is a vital hormone that mediates glucose control. Without proper insulin function, the body of a diabetic individual may develop unregulated and fluctuating glucose levels and hypo- and hyper-glycemia with severe complications that may become fatal in the absence of appropriate insulin replacement therapy. Insulin therapy has been the standard treatment for T1D for many years with increased use in treating T2D. However, insulin does not cure diabetes, and complications, such as heart and kidney disease, stroke, blindness, ulcerations and loss of extremities due to circulatory problems, gastroparesis, painful diabetic neuropathy, etc., still develop as a result of frequent wide swings in blood and tissue glucose levels despite treatment. Therefore, the challenge with insulin therapy is to supply the right amount of insulin at the appropriate times to dynamically regulate glucose levels without overcompensation.

The advent of relatively accurate blood glucose monitors has dramatically improved glucose control, but insulin therapy remains inadequate in the face of constantly fluctuating glucose challenges and metabolic needs, such as following meals or fasting periods. With multiple injections and blood glucose measurements required each day, insulin therapy is also uncomfortable and inconvenient. Insulin pumps coupled with an implanted glucose monitoring device might eventually become a valid treatment option but are currently still suboptimal. Therapeutic efforts have also been made to provide more physiologic insulin production and glucoregulation via allogeneic islet transplantation coupled with immunosuppression or immunosuppression alone. However, these approaches are severely limited at present by the difficulty in attaining sufficient donor numbers, toxic effects of immunosuppression, complications from immune deficiency, and poor results.

A need continues in the art for improved compositions and methods for the treatment of diabetes or similar diseases that are effective, achieve real-time control of glucose levels, and avoid the discomfort and dosing errors of frequent insulin applications and glucose testing.

### SUMMARY

According to a first broad aspect of the present invention, a method is provided comprising the following steps: (a) identifying an individual having or at risk of developing diabetes, pre-diabetes, or abnormal glucose metabolism; and (b) stimulating one or more intercostal or subcostal nerves derived from one or more of the following thoracic segments: T8, T9, T10, T11, and T12.

According to a second broad aspect of the present invention, a method is provided comprising the following steps: (a) identifying an individual having one or more of the following symptoms or pathological signs: elevated fasting or non-fasting glucose levels, fasting or non-fasting hyperinsulinemia, glucose intolerance, insulin resistance, dyslipidemia, or hepatic steatosis; and (b) stimulating one or more intercostal or subcostal nerves derived from one or more of the following thoracic segments: T8, T9, T10, T11, and T12.

According to a third broad aspect of the present invention, a method is provided comprising the following steps: (a) identifying an individual having or at risk of developing diabetes, pre-diabetes, or abnormal glucose metabolism; and (b) administering a composition comprising a TRPV1 agonist to the pancreas of the individual.

According to a fourth broad aspect of the present invention, a method is provided comprising the following steps: (a) identifying an individual having or at risk of developing diabetes, pre-diabetes, or abnormal glucose metabolism; and (b) administering a composition comprising a tachykinin peptide to the pancreas of the individual.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1A is a diagram showing the major branches from the sympathetic chain including the splanchnic nerves carrying nerve fibers to the pancreas;

FIG. 1B is a diagram of thoracic spinal nerves with arrow indicating an exemplary site of stimulation according to embodiments of the present invention;

FIGS. 2A through 2F are a set of fluorescent images of islets stained for DiI, glial fibrillary acidic protein (GFAP), and insulin for dye tracing following placement of DiI on spinal nerves distal to DRGs either unilaterally on left T9 through T12 spinal nerves (FIG. 2A), unilaterally on right T9 through T12 spinal nerves (FIG. 2B), bilaterally on T9 through T12 spinal nerves (FIG. 2C), or on T4 through T6 as negative control (FIG. 2D) showing that mouse T9 through T12 spinal nerves terminate in the pancreas and pancreatic lymph node with dye tracks observed in exocrine pancreas regions (FIG. 2E) and with T9 through T12 spinal nerves heavily innervating pancreatic lymph nodes;

FIG. 3A is a fluorescent image of an islet stained for DiI for dye tracing following placement of DiI on spinal nerves proximal to the site of the axotomy scar showing no DiI accumulation evidencing lack of neuronal regrowth and repair 4 weeks post surgery;

FIGS. 3B and 3C are images of serial sections of axotomy scars at 40× magnification stained with H&E and Luxol Fast Blue showing leukocytic infiltrations with marked loss of myelinated nerve bundles (FIG. 3B) and undamaged spinal nerves in control mice (FIG. 3C);

FIGS. 4A and 4B are a set of images of islets stained for GFAP (pSC), insulin (β-cells), and glucagon (α-cells) mice axotomized at 3 weeks of age showing no gross abnormalities in islet structure 5 weeks after T9-T12 axotomy in NOD.scid mice (FIG. 4A) compared to mock control NOD.scid mice (FIG. 4B);

FIG. 4C is a plot showing normal fasting blood glucose levels in axotomized (n=6) compared to mock control (n=5) mice fasted for 16 hours;

FIG. 4D is a plot showing similar body weights of axotomized (n=6) and mock control (n=5) mice at 9 weeks of age;

FIGS. 5A through 5D are a set of images of H&E stained islets from 8 week old NOD females mice receiving T9-T12 axotomy at age 3 weeks (FIG. 5A) compared to mice receiving bilateral axotomy (FIG. 5B), L1-L3 lumbar axotomy (FIG. 5C), and mock control (FIG. 5D) showing that unilateral axotomy at 3 weeks of age protects from insulitis and diabetes in NOD mice;

FIG. 5E is a plot of average insulitis scores for each group at 8 weeks of age with each point representing the mean insulitis score per individual mouse (˜100 islets/mouse) showing reduced insulitis in unilaterally T9-T12 axotomized mice;

FIG. 5F is a bar graph showing a time course of insulitis scores of axotomized versus mock control mice (n=5 mice per group);

FIG. 6 is a time course plot showing diabetes incidence for mice receiving axotomy (AX) or mock surgery at 21 days of age;

FIG. 7 is a time course plot of the percentage of NOD.scid mice that die after adoptive transfer of splenocytes from 12 week old axotomized (AX) or mock control mice;

FIGS. 8A and 8B are time course plots of blood glucose levels for NOD mice receiving T9-T12 unilateral axotomy (solid lines) or mock surgery (broken lines) showing that unilateral axotomy restores normoglycemia in new onset diabetics with each line representing an individual mouse;

FIG. 8C is a time course plot of average blood glucose levels for each group of NOD mice receiving either unilateral axotomy (thin line) or mock surgery (thick line);

FIG. 8D is a time course plot of average body weight for each group of NOD mice receiving either unilateral axotomy (thin line) or mock surgery (thick line);

FIG. 8E is a plot of fasting blood glucose levels of surviving mice 14 days after mock surgery, 14 days after axotomy (AX), or 50 days after axotomy (AX);

FIG. 9A is an image of a gel of RT-PCR samples measuring sP message levels in contralateral DRG neurons following unilateral axotomy (AX) or mock surgery relative to an actin control showing upregulation in these previously sP-negative DRG neurons;

FIGS. 9B and 9C are representative images of H&E stained DRGs showing their structure at 10× and 40× magnification;

FIGS. 9D through 9G are fluorescent images of cell bodies of contralateral T9-T12 DRG neurons following mock surgery (FIG. 9D) or unilateral axotomy (FIG. 9E) or in sP-negative (FIG. 9F) or TRPV1-negative (FIG. 9G) mice with staining for TRPV1 (upper left) and sP (lower left) provided along with a negative control (upper right) and merged image (lower right);

FIGS. 10A and 10B are plots of sP levels in the pancreas of NOD mice following T9-T12 axotomy (AX) or mock surgery compared to sP levels in the pancreas following bilateral T9-T12 axotomy (BAX) and sP levels in the heart in AX and mock treated mice as measured by ELISA at 5 weeks (FIG. 10A) or 10 weeks (FIG. 10B) after surgery with each data point representing an individual mouse;

FIGS. 11A and 11B are plots showing proliferation of BDC2.5 T cell receptor transgenic T cells labeled with CFSE and stained with antibodies against TCR Vβ4 by FACS analysis with live lymphocytes gated on forward side scatter and Vβ4 comparing mock surgery control mice (thick lines in FIGS. 11A and 11B), axotomized mice (shaded area in FIG. 11A), axotomized mice pretreated with an sP receptor antagonist (thin line in FIG. 11A), and bilateral T9-T12 axotomized mice (shaded area in FIG. 11B);

FIG. 11C is a bar graph showing a summary of data for each group of mock surgery mice, axotomized (AX) mice, axotomized mice pretreated with an sP receptor antagonist (AX-Antag); bilateral axotomized (BAX) mice, and mock surgery mice pretreated with an sP receptor antagonist (Mock Antag) expressed as a percentage of proliferating T cells (n=3-8 mice/group) showing that unilateral (AX) but not bilateral (BAX) axotomy inhibits T cell expansion in the pancreatic lymph node due to sP;

FIGS. 12A through 12F are plots showing proliferation of BDC2.5 T cell receptor transgenic T cells labeled with CFSE and stained with antibodies against TCR Vβ4 by FACS analysis with live lymphocytes gated on forward side scatter and Vβ4 for mock control mice treated with antagonist (FIG. 12F) compared to non-draining axillary lymph nodes (FIGS. 12A through 12E) used as controls (n=3-8/group);

FIG. 13 is a bar graph showing plasma extravasation in the pancreas and kidney as measured with Evans Blue dye 1 week or 8 weeks following local RTX or capsaicin treatment on T8-T11 spinal nerves relative to vehicle treatment only;

FIG. 14 is a set of images of H&E stained pancreas sections at 10× or 40× magnification after exposure of T9-T12 thoracic wall braches of spinal nerves to TRPV1 agonist (RTX) or control showing lacunar areas of regions of lymphocytic cell death;

FIG. 15 is a set of histochemical images of the pancreas showing TUNEL staining and B220 and CD3 staining following exposure of T9-T10 thoracic wall braches of spinal nerves to TRPV1 agonist (RTX) or vehicle control showing DNA strand breaks with TRPV1 agonist (RTX) treatment;

FIG. 16 is a set of images of H&E or TUNEL stained pancreatic islets following mock vehicle treatment, 6 hours after TRPV1 agonist (RTX) treatment, or 6 hours after axotomy/nerve cut (NC) showing similar but more rapid appearance of spoked wheel (H&E stain) and TUNEL-positive cell death lesions in lymphocytic islet infiltrates following TRPV1 agonist (RTX) treatment;

FIG. 17 is a set of images of TUNEL stained pancreatic lymph nodes following treatment of T9-T11 thoracic spinal nerves with TRPV1 agonist (RTX) or vehicle control or following axotomy/nerve cut (NC) of T9-T11 thoracic spinal nerves showing a rapid preponderance of TUNEL-positive cells following TRPV1 agonist (RTX) treatment;

FIG. 18 is a bar graph of the number of viable lymphocytes per lymph node (LN) following treatment of thoracic nerves with TRPV1 agonist (RTX) or vehicle alone showing depletion of lymphocytes in pancreatic lymph nodes (pLN) but axillary lymph nodes (AxLN) following TRPV1 agonist (RTX) treatment;

FIG. 19 is a bar graph (left panel) showing the absolute numbers of CD4+/Foxp3− and CD4+/Foxp3+ T cell subsets in pancreatic lymph nodes (pLN) and axillary lymph nodes (AxLN) following local treatment of thoracic nerves with TRPV1 agonist (RTX) or vehicle control and a plot (right panel) of the ratio of the CD4+/Foxp3+ to CD4+/Foxp3− T cells in pancreatic lymph nodes (pLN) showing selection of regulatory T cells following TRPV1 agonist (RTX) application with all data obtained by flow cytometry;

FIG. 20 is a density map obtained by flow cytometry gated on CD4+ T cells plotted for Foxp3 and neurokinin-1 receptor (NK1R, sP receptor) showing the absence of NK1R on Foxp3+ T cells following TRPV1 agonist (RTX) treatment;

FIGS. 21A and 21B are time course plots of blood glucose and insulin levels following intra-peritoneal (i.p.) glucose challenge (glucose tolerance test, 2 mM glucose) after fasting overnight of NOD mice receiving local treatment with TRPV1 agonist (RTX) (dotted line, n=5) or vehicle control (solid line, n=5) on thoracic spinal nerves showing that local TRPV1 agonist (RTX) treatment normalizes hyperinsulinism and insulin resistance;

FIG. 22 is a time course plot of the percentage decrease in blood glucose following insulin challenge (insulin tolerance test) in NOD mice receiving local treatment with TRPV1 agonist (RTX) (dotted line, n=5) or vehicle control (solid line, n=5) on thoracic spinal nerves showing directly that local TRPV1 agonist (RTX) treatment improves insulin sensitivity; and

FIG. 23 is a set of time course plots of blood glucose levels of individual NOD mice from separate groups of experiments following local treatment with TRPV1 agonist (RTX) (dotted line) or vehicle control (solid line) on thoracic spinal nerves showing that success rate for reversal or delay of new onset diabetes depends on blood glucose levels at time of treatment serving as a measure of residual β-cell mass.

### DETAILED DESCRIPTION

### Definitions

Where the definition of terms departs from the commonly used meaning of the term, applicant intends to utilize the definitions provided below, unless specifically indicated.

For purposes of the present invention, the terms “stimulation” or “stimulating” refer interchangeably to the triggering of a response in a desired neuron or nerve through a variety of stimulating techniques, such as by surgical, mechanical, chemical, electrical, etc., stimulation of the desired neuron or nerve. The term “stimulation” may refer to such techniques that trigger increased production and/or release of neuropeptides from pancreatic sensory afferent nerves.

For purposes of the present invention, the term “proximal” in reference to nerve tracts or paths means closer to the spinal cord than a reference point, such as the dorsal root ganglion (DRG).

For purposes of the present invention, the term “distal” in reference to nerve tracts or paths means further away from the spinal cord than a reference point, such as the dorsal root ganglion (DRG).

For purposes of the present invention, the term “ipsilateral” means on the same side of the body with respect to bilateral symmetry. For example, spinal nerves of different thoracic segments are “ipsilateral” if they are on the same side of the body (i.e., both are on the left of right side of the body).

For purposes of the present invention, the term “contralateral” means on the opposite sides of the body with respect to bilateral symmetry. For example, spinal nerves of different thoracic segments are “contralateral” if they are on the opposite sides of the body (i.e., one is on the left side and the other is on right side of the body).

For purposes of the present invention, the term “unilateral” means only on one side of the body for a given segment. For example, the term “unilateral” may mean only one of a pair of spinal nerves for a given thoracic segment of the body (i.e., only one spinal nerve on the left or the right side of the body for a given thoracic segment). However, the term “unilateral” does not refer to nerves of different segments.

For purposes of the present invention, the term “bilateral” means both sides of the body for a given segment. For example, the term “bilateral” may mean both spinal nerves for a given thoracic segment of the body (i.e., both spinal nerves on the left and the right sides of the body for a given thoracic segment). However, the term “bilateral” does not refer to nerves of different segments.

For purposes of the present invention, the term “intercostal” nerves refer to the left and right ventral ramus of the left and right spinal nerves (sometimes referred to as the ventral or anterior branch, ramus, or division) derived from thoracic segments T1 through T11. “Intercostal” nerves travel along the underside of their corresponding rib bone of their respective segment on the right and left sides. These “intercostal” nerves generally have their cell bodies in the dorsal root ganglia of their respective segments on both the right and left sides.

For purposes of the present invention, the term “subcostal” nerve refers to the ventral ramus of the left and right spinal nerves (sometimes referred to as the ventral or anterior branch, ramus, or division) derived from thoracic segment T12. “Subcostal” nerves travel along the underside of their corresponding rib bone of segment T12 on the right and left sides. These “subcostal” nerves generally have their cell bodies in the dorsal root ganglia of their respective segments on both the right and left sides.

For purposes of the present invention, the term “mammal” for embodiments of the present invention refers to the class of vertebrate animals as recognized by standard classifications. The term “mammal” may include mammals having a veterinary, agricultural, scientific, research, or medical interest. For example, a “mammal” may include rodents, such as rats, mice, etc., for use in scientific research or testing. A “mammal” may also include mammals of agricultural or veterinary interest, such as dogs, cats, pigs, cattle, sheep, goats, horses, etc. A “mammal” may include primates, such as monkeys, apes, etc., which may have veterinary or scientific research interest. Of course, a “mammal” includes humans for medical application.

For purposes of the present invention, the terms “individual,” “subject,” or “patient” refer interchangeably to a mammalian organism, such as a human, that is to be treated or stimulated according to embodiments of the present invention.

### Description

The nervous and immune systems of animals have been shown to communicate with each other through the use of neuropeptides, cytokines, or other small molecule messengers. The islets of Langerhans are comprised of α-, β-, δ- and PP cells secreting glucagon, insulin, somatostatin, and pancreatic polypeptide, respectively. Islets are densely innervated by many neurons that conglomerate into a nerve bundle termed the neuroinsular complex. See, e.g., Persson-Sjogren, S., “Neuroinsular complex type I: morphology and frequency in lean and genetically obese mice,” Pancreas 23(1):40-48 (2001); Ahren, B., “Autonomic regulation of islet hormone secretion—implications for health and disease,” Diabetologia 43(4):393-410 (2000); Kiba, T., “Relationships between the autonomic nervous system and the pancreas including the regulation of regeneration and apoptosis: recent developments,” Pancreas 29(2):e51-8 (2004), the entire contents and disclosures of which are hereby incorporated by reference. These include sympathetic and parasympathetic neurons derived from both spinal dorsal root ganglia and the vagus nerve and containing peptidergic, cholinergic, adrenergic, and GABAergic fibers. See, e.g., Lindsay, T. H. et al., “A quantitative analysis of the sensory and sympathetic innervations of the mouse pancreas,” Neuroscience 137(4):1417-26 (2006), the entire contents and disclosure of which is hereby incorporated by reference.

Pancreatic islets and associated lymph nodes in mammals are also highly innervated by primary sensory afferent neurons with their cell bodies in the dorsal root ganglia (DRGs) of thoracic segments T9 through T12 that are equally distributed between right and left sides. See, e.g. Lindsay, T. H. et al. (2006), supra. Some sensory afferent neurons also derive from the vagus nerve and nodose ganglia, but predominantly on the left side of the body. See, e.g., Fasanella, K. E., “Distribution and neurochemical identification of pancreatic afferents in the mouse,” J Comp Neurol 509:42-52 (2008), the entire contents and disclosure of which is hereby incorporated by reference. These sensory nerves may contain diverse neuron subclasses, but they prominently include sensory afferent neurons or nociceptors that express a “transient receptor potential vanilloid-1” (TRPV1, formerly VR-1) having a high activation threshold. TRPV1 is a 6-transmembrane, cation-permeable channel that functions in the sensing of various tissue insults or stimuli (e.g. nociception), such as increased temperature (e.g., 45° C. or greater), exposure to acid, changes in osmolarity, and some chemical compounds. See, e.g. Caterina, M. J. et al., “Impaired nociception and pain sensation in mice lacking the capsaicin receptor,” Science 288(5464):306-313 (2000); and Caterina, M. J., “Transient receptor potential ion channels as participants in thermo sensation and thermoregulation,” Am J Physiol Regul Integr Comp Physiol 292(1):R64-76 (2007), the entire contents and disclosures of which are hereby incorporated by reference. These stimuli may “activate” TRPV1 and result in Ca²⁺ influx through the TRPV1 channel, which may also trigger the local release of neuropeptides. Binding of agonists or activators of TRPV1 may lower the activation threshold of TRPV1, thus causing increased Ca²⁺ influx and neuropeptide release or secretion. Known TRPV1 agonists or activators include capsaicinoid compounds including capsaicin, etc., or other capsaicin analogs, such as resiniferatoxin (RTX). Other receptors on these sensory neurons may also affect (e.g. lower) the activation threshold of TRPV1. For example, insulin receptors are present on TRPV1+ sensory neurons in the pancreas and may lower the TRPV1 activation threshold when bound by insulin. Thus, the presence of other factors including TRPV1 agonists or activators, insulin, etc., may promote activation of TRPV1 channels and cause an increase in the local release of neuropeptides, such as sP and calcitonin gene related peptide (CGRP).

In addition to providing afferent or orthodromic signals toward the CNS, TRPV1+ sensory neurons also provide an efferent or antidromic function through the local release of neuropeptides, such as substance P (sP) and CGRP, at their axon terminals within the innervated tissue (e.g., pancreas). For example, Ca²⁺ influx caused by TRPV1 activation may trigger release of these bioactive neuropeptides. See, e.g. Sann, H. et al., “Efferent functions of C-fiber nociceptors,” Z Rheumatol. 57(Supp 2):8-13 (1998); and Holzer, P. et al., “Dissociation of dorsal root ganglion neurons into afferent and efferent-like neurons,” Neuroscience 86(2):389-98 (1998), the entire contents and disclosures of which are hereby incorporated by reference.

The human trpv1 gene is found on the small arm of chromosome 17 at p13.2, coordinates 3,415,493-3,447,085 (ENSEMBL assembly release 48, NCBI version 36). The mouse gene maps to chromosome 11 (band B4) at location 73,047,794-73,074,744. Human trpv1 is polymorphic with high numbers of single nucleotide polymorphisms (SNPs) identified. These SNPs may have varying effects on the functionality of TRPV1 channels and may contribute to a predisposition to diabetes or related diseases or conditions characterized by abnormal glucose regulation. There are at least six different splice variants of the human trpv1 gene with four variants utilizing alternative promoters that give rise to a protein sequence of 839 amino acids that is identical to the canonical or wild-type TRPV1 protein. There are other alternative splice variants of the trpv1 gene that differ in the length of mature protein, varying from 510 to 849 amino acid residues. The trpv1 genes of mouse and rat use at least three different promoters. Such diversity in promoters may play a role in the regulation of expression and function of TRPV1 splice variants in different tissues.

Substance P (sP) is an eleven amino acid (Arg-Pro-Lys-Pro-Gln-Gln-Phe-Phe-Gly-Leu-Met) tachykinin neurotransmitter, first identified as a pain-signaling neuropeptide, but mediating several other non-neuronal functions. See, e.g., O'Connor, T. M. et al., “The role of substance P in inflammatory disease,” J Cell Physiol 201(2): 167-80 (2004); and Reinke, E. et al., “Breaking or making immunological privilege in the central nervous system: the regulation of immunity by neuropeptides,” Immunol Lett 104(1-2): 102-109 (2006). Neuronal expression of sP is largely restricted to small dorsal root ganglion neurons, such as TRPV1+ nociceptors, but expression is not entirely restricted to neurons.

Generally, sP can act on non-neuronal cells which express the major sP receptor, neurokinin 1 receptor (NK1R), under certain physiological circumstances. The NK1R receptor is a seven membrane-spanning guanine nucleotide binding, G protein-coupled receptor, with conserved sequence (about 95%) between mouse and human. See, e.g. Hershey, A. D. et al., “Molecular and genetic characterization, functional expression, and mRNA expression patterns of a rat substance P receptor,” Ann NY Acad Sci 632:63-78 (1991); Hershey, A. D. et al., “Molecular characterization of a functional cDNA encoding the rat substance P receptor,” Science 247(4945):958-62 (1990); and Nakanishi, S., “Mammalian tachykinin receptors,” Annu Rev Neurosci 14:123-36 (1991), the entire contents and disclosures of which are hereby incorporated by reference. Binding of sP to the receptor initiates internalization of the peptide/receptor complex, which may result in desensitization of cells to sP signaling as a mode of sP stimulus regulation. Downstream signaling from NK1-R activates phospholipase C (PLC) leading to formation of IP3 and DAG, calcium mobilization, and activation of protein kinase C (PKC). See, e.g., O'Connor, T. M. et al. (2004), supra. In a T cell, this may ultimately lead to T cell activation or activation-induced cell death (AICD) depending on the circumstances and level of activation.

As mentioned, Type-1 diabetes (T1D) is an autoimmune disease governed by multiple genetic and environmental risk factors. Overt T1D typically reflects glucose intolerance due to insulin deficiency. It is the end result of pre-diabetes, with progressive lymphoid infiltration around and then inside pancreatic islets of Langerhans with subsequent destruction of insulin-producing β-cells by autoreactive T lymphocytes. See, e.g., Anderson, B. et al., “The NOD mouse: a model of immune dysregulation,” Annu Rev Immunol 23:447-485 (2005), the entire contents and disclosure of which is hereby incorporated by reference. T1D is characterized by a permissive immune system that fails to impose tolerance to arrays of self-antigens. Although the initiating events are not fully understood, β-cell stress and β-cell death in the course of early islet restructuring are thought to provide sensitizing autoantigens which expand autoreactive T cell pools in pancreatic lymph nodes.

Self-antigens targeted in T1D are expressed by β-cells and, in most cases, elsewhere in the body. Given their presence in other tissues, it has been unclear why T cells infiltrate only islets and their associated glia in T1D. It has also been unclear whether autoimmunity and islet inflammation are related to hyperinsulinism and insulin resistance. Both hyperinsulinism and insulin resistance are observed in pre-diabetic humans and non-obese diabetic (NOD) mice, which are used as a model for T1D, even at a young age. See, e.g. Amrani, A. et al., “Glucose homeostasis in the nonobese diabetic mouse at the prediabetic stage,” Endocrinology 139:1115-1124 (1998); and Chaparro, R. J. et al., “Nonobese diabetic mice express aspects of both type 1 and type 2 diabetes,” PNAS USA 103:12475-12480 (2006), the entire contents and disclosures of which are hereby incorporated by reference.

Functional interactions between the nervous and immune systems are known, but connections between islet autoimmunity and the nervous system have remained ill defined. See, e.g. Carillo, J. et al., “Islet-infiltrating β-cells in nonobese diabetic mice predominantly target nervous system elements,” Diabetes 54:69-77 (2005), the entire contents and disclosure of which is hereby incorporated by reference. Although hyperinsulinemia and reduced insulin sensitivity have been shown to adversely affect sensory nerve function, the mechanism underlying this observation has not been clearly understood. See, e.g., Delaney, C. A. et al., “Insulin sensitivity and sensory nerve function,” Clin Exp Neurol 31:19-37 (1994). Based on the present work, homing of T cells to the islet may be viewed as a response to islet stress caused by a hypofunctional TRPV1. Pancreatic islets are innervated by meshworks of TRPV1+ primary sensory neurons, but their local function is unclear. See, e.g. Ahren, B. (2000), supra.

In studies using non-obese diabetic (NOD) mice, the present inventors show that TRPV1+ sensory afferent neurons play a fundamental role in β-cell function and diabetes pathoetiology. NOD mice develop a T1D-like disease with islet destruction resulting from T-cell infiltration and subsequent insulin deficiency. NOD mice are shown to have mutations in the trpv1 gene, resulting in a hypo-functional and under-expressed TRPV1 protein having two amino acid substitutions of conserved residues. NOD mice are shown to have depressed nociceptive responses and edema in response to intradermal capsaicin administration, and the maximum recorded Ca²⁺ response to capsaicin in the dorsal root ganglion (DRG) is significantly reduced in NOD mice relative to controls. However, KCl-evoked Ca²⁺ responses are not different between NOD and control mice suggesting specificity to TRPV1. TRPV1+ neurons in NOD mice are shown to be deficient in their release of efferent neuropeptides in the pancreatic islet, and β-cell stress and subsequent autoimmune infiltration of T-cells into the islet results. Congenic replacement of the Idd4 locus encompassing the trpv1 mutant gene with the homologous genomic interval from a C57/BL6J (B6) mouse protects from insulitis and the development of diabetes, although splenocytes from these congenic animals retain the ability to transfer both insulitis and diabetes to immune-deficient NOD.scid mice. See, e.g., Razavi, R. et al., “TRPV1+ Sensory Neurons Control β Cell Stress and Islet Inflammation in Autoimmune Diabetes,” Cell 127:1123-1135 (2006); and Tsui, H. et al., “‘Sensing’ autoimmunity in type 1 diabetes,” TRENDS in Mol. Med. 13(10):405-413 (2007); Tsui, H. et al., “Neuronal elements in the pathogenesis of type 1 diabetes,” Rev Endocr Metab Disord 4(3):301-310 (2003), the entire contents and disclosures of which are hereby incorporated by reference.

To investigate the role of islet innervation by TRPV1+ primary afferent sensory neurons in T1D pathogenesis, the present work uses neonatal treatment of diabetes-prone NOD mice with capsaicin to permanently remove these neurons. See, e.g. Caterina, M. J. et al., “The vanilloid receptor: a molecular gateway to the pain pathway,” Ann Rev Neurosci 24:487-517 (2001); and Jansco, G. et al., “Pharmacologically induced selective degeneration of chemosensitive primary sensory neurons,” Nature 270(5639):741-43 (1977), the entire contents and disclosures of which are hereby incorporated by reference. As expected, capsaicin-treated NOD (NOD^{caps}) mice are viable, fertile, and without abnormalities in their growth or gross tissue structure including the pancreas. Islet infiltrations by T-cells in most NOD^{caps} mice are significantly reduced compared with NOD^{ctrl} mice and entirely absent in a third of the mice. Strikingly, there is little of the typical insulitis progression over time in NOD^{caps} mice. Correspondingly, neonatal capsaicin treatment delayed the onset of diabetes and reduced its incidence. Thus, reduced neuropeptide release appears to have a deleterious effect in NOD^{caps} mice, but positive outcomes are observed either with normal neuropeptide concentrations (as in wild-type mice) or with removal of TRPV1+ afferent neurons and their associated neuropeptides.

Capsaicin treatment does not have a general affect on autoimmune infiltrations since NOD^{caps} mice still exhibit a Sjögren-like disease with submandibular lymphocyte infiltrates that is under separate genetic control in NOD mice. Furthermore, capsaicin treatment did not affect general immune function or development in NOD mice since systemic T-cells pools autoreactive with disease-associated antigens (e.g., insulin, GA065, GFAP, S100b, HSP60, BSA) are indistinguishable between NOD^{caps} and NOD^{ctrl} mice, and the number of circulating diabetogenic CD8+ T-cells that recognize an islet-specific antigen (NRP-V7) is similar in NOD^{caps} and NOD^{ctrl} mice. Delayed type hypersensitivity reactions developed normally in NOD^{caps} mice, suggesting maintenance of antigen presentation function and effector T cell generation. Furthermore, NOD^{caps} mice that did develop disease showed insulitis, and spleen cells from NOD^{caps} and NOD^{ctrl} mice equally transfer T1D-like disease with normal kinetics to lymphocyte-free NOD.scid recipients that are not treated with capsaicin. The fact that NOD^{caps} mice retain loss of self-tolerance with islet-reactive T cell pools in NOD^{caps} mice that transfer insulitis to NOD.scid recipients, clearly separates autoreactivity from autoimmune disease: only the latter involves a hypofunctional TRPV1 and reduced neuropeptide release from TRPV1+ sensory neurons. See, e.g. Tsui, H. et al., “‘Sensing’ autoimmunity in type 1 diabetes,” Trends Mol Med, 13(10):405-13 (2007), the entire contents and disclosure of which is hereby incorporated by reference.

In contrast to untreated NOD mice, pancreatic NOD^{caps} lymph node tissue contains significantly reduced proportions and absolute numbers of CD8+ and activated CD8+/CD69+ effector T lymphocytes that are critical for islet destruction. As a hallmark of pre-diabetes progression, pre-diabetic NOD mice selectively lose CD4+/CD25+ and Foxp3+ regulatory T cell subsets in pancreatic lymph node tissue. However, NOD^{caps} mice maintained their regulatory T cell compartment in pancreatic lymph nodes beyond 12-16 weeks of age. Thus, there are significant differences in the local immune system in the pancreas of NOD^{caps} and NOD^{ctrl} mice, which is consistent with the suppression of chronic progressive islet inflammation in these animals.

Low dose cyclophosphamide accelerates NOD diabetes by multiple mechanisms. Consistently, low dose cyclophosphamide accelerates diabetes development in both NOD^{caps} and NOD^{ctrl} mice and is associated with reversal of the regulatory T cell maintenance in NOD^{caps} versus NOD^{ctrl} pancreatic lymph nodes. Thus, NOD^{caps} mice retain the principal ability to generate diabetogenic T cell pools, and loss of self-tolerance and target tissue invasion appear to be separate and distinct elements of T1D pathogenesis with TRPV1+ sensory neurons playing a critical role in the accumulation of immune cells in the pancreas.

Abnormal TRPV1 function might selectively lead to islet pathology if there is a local disease-predisposing TRPV1-based effect on β-cell function and if that effect is removed in NOD^{caps} mice. The insulin-rich islet milieu represents a unique environment for TRPV1+ sensory nerve terminals, as they express insulin receptors and insulin sensitizes and lowers the activation threshold of TRPV1 channels. See, e.g., Van Buren, J. J. et al., “Sensitization and translocation of TRPV1 by insulin and IGF-1,” Mol Pain 1(1):17 (2005), the entire contents and disclosure of which is hereby incorporated by reference. Based on diminished capsaicin-evoked neurogenic inflammation and reduced TRPV1 expression and function in NOD mice, neuropeptides, such as sP, may be mediators of neurogenic inflammation, and their local release from the peripheral terminals of sensory neurons may be depressed in NOD mice. Consistent with its reduced release, levels of sP are elevated in NOD dorsal root ganglia, the location of substance P synthesis, presumably due to their accumulation, and the pancreas of NOD mice shows accumulation of more sP in nerve endings. This is not due to inflammation as it was also observed in NOD.scid mice.

Based on these findings, if depressed sP release is critical for NOD islet pathology, then increasing pancreatic sP levels by local injection or infusion (e.g., intra-arterial (i.a.) injection or infusion) of sP into the pancreas is predicted to relieve the pathogenic process. Unlike systemic intravenous (i.v.) injection, after 2 days following i.a. injection of sP (e.g., 2 nmoles per kg body weight) into the pancreas of prediabetic NOD animals, it is shown that about 80% of all islets are free of T cell infiltration in these animals with only a small residual infiltrate in the remainder. Following sP administration, and without insulin therapy, over half of the i.a. injected NOD animals normalize blood glucose levels. In responding mice, fasting blood glucose returns to near normal levels rapidly and remains at these levels for about 2 to 8 weeks. Furthermore, sP administration dramatically enhances insulin sensitivity, suggesting that the elevated insulin resistance at diagnosis is normalized. On average, mice that reverse diabetes have less extreme hyperglycemia at the time of diagnosis (i.e., prior to treatment) than non-responding mice, likely reflective of a larger residual β-cell mass at the time of sP administration. However, even in mice that fail to reverse hyperglycemia, sP administration causes a significant improvement of metabolic control, preventing the progressive loss of body weight typical of overtly diabetic NOD mice. This improvement corresponds to significantly improved insulin sensitivity which enhances the effectiveness of the small remaining β-cell mass at diabetes onset. By contrast, blood glucose rises progressively, body weights decline, and animals are sacrificed because of severe diabetes between days 12-16 in all vehicle-injected (i.e., without sP) control animals similarly to untreated NOD mice.

Taken together, these data show that reduced neuropeptide release by pancreatic TRPV1+ nerve terminals is a pathogenic event in NOD diabetes that may be amenable to therapeutic correction. This conclusion is supported by the fact that either removal of TRPV1+ neurons or local intra-arterial injection of sP into the pancreas of NOD mice evidenced similar positive results. Indeed, pancreatic sP injection normalized all parameters tested: clearing of insulitis lesions, enhancement of insulin sensitivity, and consequent reversal of overt diabetes that lasted for a period of weeks.

One possible target for sP is activated pancreatic T cells since these cells express the NK1R receptor for sP. See, e.g., Zhang, Y. et al., “Tachykinins in the immune system,” Curr Drug Targets 7(8):1011-1120 (2006); and Persson-Sjogren, S. et al., “Expression of the NK-1 receptor on islet cells and invading immune cells in the non-obese diabetic mouse,” J Autoimmun 24(4):269-79 (2005), the entire contents and disclosures of which are hereby incorporated by reference. NK1R expression is detected on a portion of the T cells from pancreatic lymph nodes, but upon in vitro activation with Concanavalin A (Con A), essentially all NOD splenic T cells expressed NK1R. To determine the functional effect of NK1R ligation, the sP response of activated CD4+ NOD T cells in vitro is tested. sP is shown to abrogate cell proliferation and survival of these cells in a dose-dependent fashion. In addition, injection of sP into the pancreas reduces cellularity and clonal expansion of BDC2.5 T cells in vivo, and BDC2.5 cells pretreated with sP are less able to expand in pancreatic lymph nodes. Other than affecting the proliferation or survival T cells, sP may also affect the immigration or residence of T cells in pancreatic tissue.

β-cell stress has been suggested as an early element or trigger of T1D pathoetiology. Therefore, it is important to determine whether hypofunctional TRPV1 in NOD mice is related to observed signs of β-cell stress, hyperinsulinism, and abnormal glucose clearance. High normal glucose levels observed after standard i.p. glucose challenge in NOD.scid^{ctrl} mice is significantly reduced in NOD.scid^{caps} mice, and the improved NOD.scid^{caps} glucose response is associated with significantly less insulin production, suggesting more effective insulin action (i.e., insulin sensitivity) with removal of TRPV1+ sensory neurons.

B6 mice develop elevated insulin resistance and a metabolic T2D-like disease with diet induced obesity (DIO) that is attributed to the functional deletion of nicotinamide transhydrogenase. Consistently, high blood glucose levels are observed in B6 mice after standard i.p. glucose challenge. However, B6.TRPV1^{-/-} mice show a significantly improved glucose response analogous to NOD^{caps} mice, further pointing to the possibility that TRPV1 may play a general role in β-cell physiology.

To more directly assess if these observations in B6.TRPV1^{-/-} mice reflect enhanced insulin sensitivity due to TRPV1 removal, glucose clearance after a single insulin injection is measured. Compared to their respective control animals, NOD^{caps} and B6.TRPV1^{-/-} mice show significantly enhanced and accelerated glucose clearance, thus providing evidence for reduced insulin resistance due to the absence of TRPV1 in these two independent mouse models. Enhanced insulin resistance associated with mutant TRPV1 in NOD mice may cause a persistent β-cell stress, likely worsening with progressive islet inflammation. The present studies in B6 mice indicate that TRPV1 and TRPV1+ sensory neurons may broadly impact insulin and glucose homeostasis including insulin resistance. Therefore, therapeutic compositions or methods targeting TRPV1+ neurons or causing release of neuropeptides may be used to treat different types of diabetes, including both type 1 and type 2 diabetes despite their differing pathoetiologies.

TRPV1 emerges as a central controller of pancreatic islet stress and T cell infiltration leading to islet destruction and insulin deficiency. The present findings challenge the view that diabetes is due solely to immunological and endocrine abnormalities. Rather, it is shown that the nervous system and particularly TRPV1+ primary sensory neurons have a critical role in diabetes progression. In addition, evidence shows that TRPV1 function further influences insulin sensitivity in different mouse models, suggesting that removal of TRPV1+ neurons or normalization of TRPV1 function, such as by neuropeptide administration or by stimulation of TRPV1+ neurons, may have therapeutic potential in treating T2D or other types of diabetes in addition to T1D that are characterized by insulin resistance. Elimination of TRPV1+ neurons by neonatal capsaicin treatment, transient functional normalization of sP levels in the pancreas by acute local sP injection, and replacement of mutant TRPV1 with a wild-type copy in Idd4 congenic animals all have similar outcomes: abrogation of insulitis and normalization of insulin sensitivity and glucose metabolism. These outcomes are observed despite the unimpeded generation of potentially autoreactive T lymphocytes in these “rescued” mice that can transfer disease to untreated NOD.scid hosts.

Without being bound by any theory, it is proposed that there may be a local feedback interaction between β-cells and the primary sensory neurons innervating the islets. According to this model, insulin present in the islet milieu may ligate insulin receptors on TRPV1+ sensory afferent islet terminals to lower the activation threshold of TRPV1 with subsequent Ca²⁺ influx and local release of neuropeptides (e.g., sP, CGRP). Normally, this interaction or feedback loop is in balance with appropriate levels of neuropeptide secretion avoiding β-cell stress and subsequent proliferation and/or infiltration of autoreactive T-cells into the islet. However, in the NOD mouse, hypofunctional TRPV1 unbalances this feedback, thus leading to β-cell stress and subsequent proliferation and/or infiltration by autoreactive T cell pools. Removing TRPV1+ neurons in NOD mice eliminates the unbalanced, pathogenic interaction, while administering sP exogenously to NOD mice re-normalizes the interaction at least transiently.

For further description of the present work, see, e.g., Razavi, R. et al. (2006), supra; Tsui, H. et al. (2007), supra; Tsui, H. et al., “Neuronal elements in the pathogenesis of type 1 diabetes,” Rev Endocr Metab Disord 4(3):301-310 (2003); and U.S. patent application Ser. Nos. 11/638,830 and 12/394,261, the entire contents and disclosures of which are hereby incorporated by reference.

According to a broad aspect of the present invention, one or more neuropeptide(s) may be administered to an individual, subject, or patient having or at risk of developing diabetes or abnormal glucose metabolism. According to embodiments of the present invention, such a neuropeptide may include a tachykinin peptide as understood in the art, such as substance P (sP), neurokinin A, neurokinin K, neuropeptide gamma, neurokinin B, etc., or a precursor thereof. The polypeptide sequences of these tachykinin peptides are known in the art. Such a neuropeptide or tachykinin peptide may include any peptide that binds to a known mammalian tachykinin receptor, such as a NK-1 receptor (NK1R), etc. According to some embodiments, a pharmaceutical composition comprising a neuropeptide or a tachykinin peptide in combination with a pharmaceutically acceptable carrier may be administered to an individual, subject, or patient having or at risk of developing diabetes or abnormal glucose metabolism. According to some embodiments, the pharmaceutical composition may comprise a therapeutically effective amount of one or more neuropeptide(s) or tachykinin peptide(s) in combination with a pharmaceutically acceptable carrier.

According to some embodiments of the present invention, a neuropeptide or a tachykinin peptide, such as sP, or a pharmaceutical composition comprising a neuropeptide or tachykinin peptide, such as sP, and a pharmaceutically acceptable carrier, may be administered to the pancreas of an individual, subject, or patient having or at risk of developing diabetes or abnormal glucose metabolism. For example, a neuropeptide or a tachykinin peptide, such as sP, or a pharmaceutical composition comprising a neuropeptide or a tachykinin peptide and a pharmaceutically acceptable carrier may be administered by intra-arterial (i.a.) injection into the pancreas of an individual, subject, or patient having or at risk of developing diabetes or abnormal glucose metabolism.

According to another broad aspect of the present invention, one or more agonist(s) or activator(s) of a mammalian transient receptor potential vanilloid-1 (TRPV1) channel are administered to an individual, subject, or patient having or at risk of developing diabetes or abnormal glucose metabolism. According to embodiments of the present invention, such a TRPV1 agonist or activator may include a capsaicinoid compound, such as capsaicin, dihydrocapsaicin, nordihydrocaposaicin, homodihydrocapsaicin, homocapsaicin, etc., or other capsaicin analogs, such as resiniferatoxin (RTX), etc. The chemical structures of these compounds are known in the art. According to some embodiments, a pharmaceutical composition comprising a TRPV1 agonist or activator, such as a capsaicinoid compound or capsaicin analog, in combination with a pharmaceutically acceptable carrier may be administered to an individual, subject, or patient having or at risk of developing diabetes or abnormal glucose metabolism. According to some embodiments, the pharmaceutical composition may comprise a therapeutically effective amount of one or more TRPV1 agonist(s) or activator(s), such as a capsaicinoid compound and/or capsaicin analog, in combination with a pharmaceutically acceptable carrier.

According to some embodiments of the present invention, one or more TRPV1 agonists or activators, such as capsaicinoid compounds or capsaicin analogs, or a pharmaceutical composition comprising one or more TRPV1 agonists or activators, such as capsaicinoid compounds or capsaicin analogs, and a pharmaceutically acceptable carrier may be administered to the pancreas of an individual, subject, or patient having or at risk of developing diabetes or abnormal glucose metabolism. For example, one or more TRPV1 agonists or activators, such as capsaicinoid compounds or capsaicin analogs, or a pharmaceutical composition comprising one or more TRPV1 agonists or activators, such as capsaicinoid compounds or capsaicin analogs, and a pharmaceutically acceptable carrier may be administered by intra-arterial (i.a.) injection into the pancreas of an individual, subject, or patient having or at risk of developing diabetes or abnormal glucose metabolism.

Although administration of one or more neuropeptides or TRPV1 agonists or activators to the pancreas is a promising technique for treating an individual, subject, or patient having or at risk of developing diabetes, such administration may require repeated administrations or injections to the pancreas to effectively treat the disease. Therefore, other methods of achieving stimulation of pancreatic sensory afferent nerves are also explored.

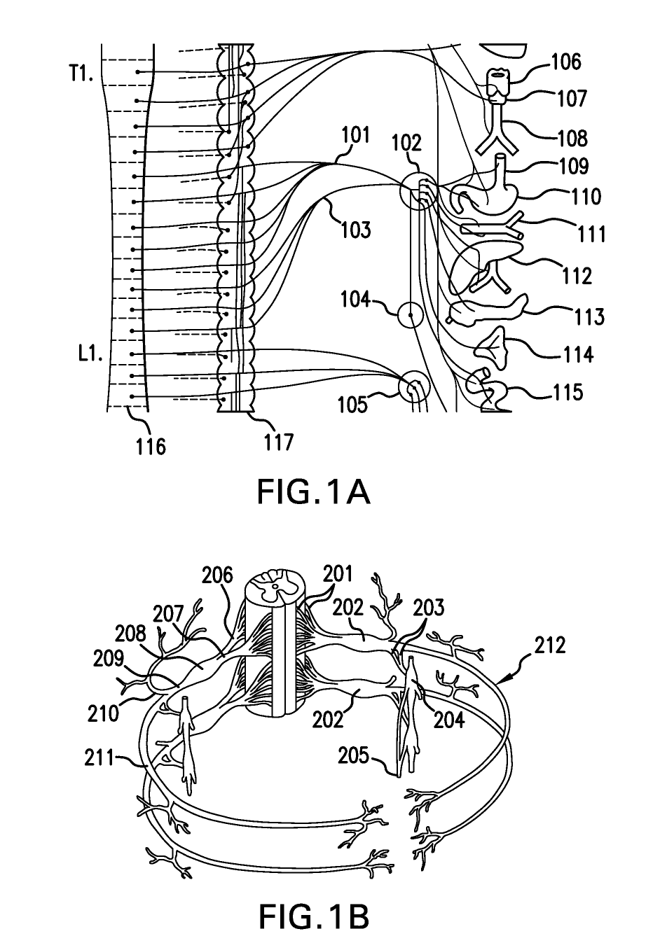

FIG. 1A shows the major branches from the sympathetic chain including the splanchnic nerves carrying nerve fibers to the pancreas with the great splanchnic nerve 101, celiac ganglion 102, small splanchnic nerve 103, superior mesenteric ganglion 104, inferior mesenteric ganglion 105, larynx 106, trachea 107, bronchi 108, esophagus 109, stomach 110, blood vessels 111 of abdomen, liver and ducts 112, pancreas 113, adrenal 114, small intestine 115, spinal cord 116, and sympathetic chain 117.

FIG. 1B is a diagram of thoracic spinal nerves showing rootlets 201, dorsal root ganglion (DRG) 202 and 208, sympathetic rami 203, ganglion of sympathetic chain 204, roots of splanchnic nerve 205, dorsal root of spinal nerve 206, ventral root of spinal nerve 207, spinal nerve 209, dorsal ramus of spinal nerve 210, and ventral ramus of spinal nerve 211. Arrow 212 in FIG. 1B indicates an exemplary site of stimulation according to embodiments of the present invention.

Pancreatic TRPV1+ sensory neurons have their cell bodies in the dorsal root ganglia (DRGs) of thoracic segments T8 through T12 (i.e., T8, T9, T10, T11, and/or T12) near the spinal cord. Pancreatic TRPV1+ sensory neurons generally travel along the splanchnic nerve paths or branches to reach the spinal cord (see FIG. 1A and sympathetic rami 203, ganglion of sympathetic chain 204 and roots of splanchnic nerve 205 of FIG. 1B).

Intercostal and/or subcostal nerves from segments T8 through T12 (i.e., T8, T9, T10, T11, and/or T12) containing somatosensory neurons also have their cell bodies in the same DRGs of these thoracic segments. (See FIG. 1B) Therefore, it is proposed that stimulation of one or more of these intercostal and/or subcostal nerves of spinal nerves derived from thoracic segments T8 through T12 (i.e., T8, T9, T10, T11, and/or T12) may potentially impact the functioning of pancreatic TRPV1+ sensory neurons indirectly since they have their respective cell bodies in the same DRGs. As described below, it is shown that unilateral stimulation of intercostal and/or subcostal nerves from thoracic segments T9 through T12 in mice by axotomy or chemical (e.g., RTX) treatment results in reduced populations of infiltrating lymphocytes and associated insulitis in the pancreas, normalization of elevated insulin resistance and glucose levels, increased sP expression, improved survival, and/or reversal of diabetes symptoms in these animals presumably via indirect stimulation of pancreatic sensory afferent neurons.

According to a broad aspect of the present invention, stimulation or activation of intercostal and/or subcostal nerves derived from thoracic segments T8 through T12 (i.e., T8, T9, T10, T11, and/or T12) may be achieved through a variety of different approaches. According to some embodiments, stimulation of intercostal and/or subcostal nerves may be achieved through exposure of these nerves to chemical compounds. According to other embodiments, stimulation of intercostal and/or subcostal nerves may be achieved through exposure of these nerves to electrical signals or impulses. According to some embodiments, stimulation of intercostal and/or subcostal nerves may also be achieved by mechanical or surgical techniques, such as by pulling, tugging, agitating, cutting, etc. of these nerves.

For purposes of illustration, FIG. 1B shows a simplified view of an example of two adjacent sets of spinal of thoracic spinal nerves. The exemplary features shown in FIG. 1B are similar between spinal nerves of the different thoracic segments. Therefore, these features may be generalized for each of the thoracic segments of spinal nerves. Generally speaking, the dorsal roots 206 and ventral roots 207 of spinal nerves within each thoracic segment exit the spinal cord and merge to form the thoracic spinal nerves 209, and the dorsal root ganglia (DRGs) 208 are located in the dorsal root 206 on both sides of each segment. The thoracic spinal nerves 209 branch to form the dorsal ramus (sometimes referred to as the dorsal or posterior branch, ramus, or division) 210 and the ventral ramus (sometimes referred to as the ventral or anterior branch, ramus, or division) 211 of the thoracic spinal nerves 209 on each side of the spinal cord. The dorsal rami 210 of the thoracic spinal nerves are generally smaller than the ventral rami 211 and innervate the muscles and skin of the back, while the ventral rami 211 of thoracic spinal nerves are generally larger and travel along the underside of the corresponding rib bone of each thoracic segment to innervate tissue generally on the ventral side of the trunk. The ventral rami 211 of spinal nerves 209 that are derived from the thoracic segments on the right and left side may also be referred to as intercostal nerves, whereas the ventral rami 211 of the spinal nerve 209 derived from the thoracic segment T12 on the right and left side may also be referred to as subcostal nerves. Arrow 212 in FIG. 1B indicates an exemplary site of stimulation according to embodiments of the present invention. However, the site of stimulation may be at any position or location along the length of the intercostal and/or subcostal nerve(s) to be treated but may preferably be near the DRG of the respective nerve.

According to embodiments of the present invention, intercostal and/or subcostal nerves derived from thoracic segments T8 through T12 (i.e., T8, T9, T10, T11, and/or T12) of an individual, subject, or patient having or at risk of developing diabetes or abnormal glucose metabolism may be activated or stimulated by chemical compounds, electrical signals or impulses, mechanical or surgical techniques, etc. According to some embodiments, the intercostal and/or subcostal nerves derived from thoracic segments T8 through T12 (i.e., T8, T9, T10, T11, and/or T12) may be stimulated at any location or position along the length of one or more of these nerves distal to the dorsal root ganglion (DRG) of each spinal nerve stimulated. According to some embodiments, the intercostal and/or subcostal nerves derived from thoracic segments T8 through T12 (i.e., T8, T9, T10, T11, and/or T12) may be stimulated at any location or position along the length of one or more of these nerves (i.e., ventral rami of the spinal nerves at these segments) distal to the branching point of the dorsal ramus of the spinal nerve for each spinal nerve stimulated. According to some embodiments, the intercostal and/or subcostal nerves derived from thoracic segments T8 through T12 (i.e., T8, T9, T10, T11, and/or T12) may preferably be stimulated at a location or position near the DRG of each spinal nerve stimulated, which may provide a more robust response.

According to embodiments of the present invention, regardless of the mode, type, or technique of activation or stimulation (i.e., mechanical, surgical, chemical, electrical, etc.), one or more intercostal and/or subcostal nerves derived from thoracic segments T8 through T12 (i.e., T8, T9, T10, T11, and/or T12) of an individual, subject, or patient having or at risk of developing diabetes or abnormal glucose metabolism may be activated or stimulated unilaterally for each of the segment(s) of the one or more intercostal and/or subcostal nerves to be treated. Although bilateral stimulation of spinal nerves derived from a given segment may be useful or beneficial in some circumstances, stimulation of spinal nerves derived from each of the thoracic segments T8 through T12 that are to be treated may generally be performed unilaterally. Without being bound by any theory, it is believed that unilateral stimulation allows for a beneficial response via contralateral sensory afferent neurons through an unknown mechanism that may involve the higher orders of the central nervous system (CNS), which may be diminished or eliminated with certain types of bilateral stimulation. However, although stimulation may generally be performed unilaterally at each of the one or more segment(s) of spinal nerves receiving stimulation, stimulation of two or more intercostal and/or subcostal nerves of different segments may each be performed unilaterally on different sides of the spinal cord (i.e., between the different segments). Thus, stimulation of any combination of one or more intercostal and/or subcostal nerves derived from thoracic segments T8 through T12 (i.e., T8, T9, T10, T11, and/or T12) of an individual, subject, or patient having or at risk of developing diabetes or abnormal glucose metabolism is contemplated, but unilateral stimulation is generally preferred at each segment albeit unilateral stimulation may be performed on different sides of the spinal cord between different segments. According to some embodiments of the present invention, each of the modes, types, or techniques of neuronal stimulation (i.e., mechanical, surgical, chemical, electrical, etc.) may require surgical incision, injection, catheterization, implantation, etc., to access the intercostal and/or subcostal nerves to be stimulated.

According to embodiments of the present invention, one or more intercostal and/or subcostal nerves of spinal nerves derived from thoracic segments T8 through T12 (i.e., T8, T9, T10, T11, and/or T12) of an individual, subject, or patient having or at risk of developing diabetes or abnormal glucose metabolism may be stimulated by exposure of one or more of these intercostal and/or subcostal nerves to one or more TRPV1 agonist(s) or activator(s). In other words, one or more TRPV1 agonist(s) or activator(s) may be administered to one or more intercostal and/or subcostal nerves of spinal nerves derived from thoracic segments T8 through T12 (i.e., T8, T9, T10, T11, and/or T12) of an individual, subject, or patient having or at risk of developing diabetes or abnormal glucose metabolism. According to embodiments of the present invention, such a TRPV1 agonist or activator may include one or more capsaicinoid compounds, such as capsaicin, dihydrocapsaicin, nordihydrocaposaicin, homodihydrocapsaicin, homocapsaicin, etc., and/or one or more other capsaicin analogs, such as resiniferatoxin (RTX), etc.

According to some embodiments, a pharmaceutical composition comprising a TRPV1 agonist or activator in combination with a pharmaceutically acceptable carrier may be administered to one or more intercostal and/or subcostal nerves of spinal nerves derived from thoracic segments T8 through T12 (i.e., T8, T9, T10, T11, and/or T12) of an individual, subject, or patient having or at risk of developing diabetes or abnormal glucose metabolism. The pharmaceutical composition may comprise a therapeutically effective amount of one or more TRPV1 agonist(s) or activator(s) in combination with a pharmaceutically acceptable carrier.

To administer a TRPV1 agonist or activator to one or more intercostal and/or subcostal nerves of an individual, subject, or patient having or at risk of developing diabetes or abnormal glucose metabolism according to some embodiments of the present invention, one or more surgical incision(s) at a desired location(s) may be needed to allow access to the one or more intercostal and/or subcostal nerves that are to be treated. Alternatively, according to some embodiments, a TRPV1 agonist or activator may be administered to one or more intercostal and/or subcostal nerves of an individual, subject, or patient having or at risk of developing diabetes or abnormal glucose metabolism by one or more injection(s), such as by use of a syringe, etc., which may be made at one or more desired location(s). According to embodiments of the present invention, a TRPV1 agonist or activator, or a pharmaceutical composition comprising the TRPV1 agonist or activator, may be administered at a desired location(s) such that the TRPV1 agonist or activator is placed in direct contact with or adjacent to the one or more intercostal and/or subcostal nerves of thoracic segments T8 through T12 (i.e., T8, T9, T10, T11, and/or T12). As stated above, the one or more surgical incisions, injections, etc., may be made at one or more location(s) to allow access to one or more intercostal and/or subcostal nerves to be treated with a TRPV1 agonist or activator at a position along the length of these nerves that is preferably near (but distal to) their respective DRG. Regardless of the number of intercostal and/or subcostal nerve(s) treated or the number of location(s) of surgical incision(s) or injection(s) performed, the individual, subject, or patient may be treated one or more times at each location, with each treatment being done either closely together in time, such as within minutes or hours, or separately over a longer period of time, such as days, weeks, months, years, etc., as part of a treatment regimen or individually in response to disease progression.

According to embodiments of the present invention, an individual, subject, or patient having or at risk of developing diabetes or abnormal glucose regulation or metabolism may be defined as a mammal, such as a human, having diabetes, pre-diabetes, metabolic syndrome (MetSyn), or any other condition associated with or characterized by abnormal glucose regulation or metabolism. For example, an individual, subject, or patient having or at risk of developing diabetes or a condition characterized by abnormal glucose regulation or metabolism may include any individual, subject, or patient having or at risk of developing one or more of the following diseases or conditions according to standard clinical, medical, and/or pathological criteria: type 1 diabetes (T1D), type 2 diabetes (T2D), type 1.5 diabetes, gestational diabetes, type 3 diabetes (T3D), Latent Autoimmune Diabetes of the Adult (LADA), impaired glucose tolerance (IGT, biochemical diabetes), impaired fasting glucose (IFG), etc., as understood by a skilled artisan, such as a physician, endocrinologist, veterinarian, etc., as the case may be. As stated above, the present study supports the conclusion that modification of TRPV1 function in pancreatic TRPV1+ sensory neurons may improve (i.e., reduce) insulin resistance as well as avoid destruction of β-cells in the islet. Thus, modification of TRPV1+ neurons and/or neuropeptide release from sensory afferent neurons may provide a way to manage or treat diabetes types other than T1D, such as T2D, etc., that are classically more associated with insulin resistance rather than insulin deficiency.

According to some embodiments, an individual, subject, or patient having or at risk of developing diabetes or abnormal glucose regulation or metabolism may be an individual, subject, or patient having any of the known clinical symptoms or pathological signs commonly associated with diabetes or prediabetes according to the judgment of a skilled artisan, such as a physician, endocrinologist, veterinarian, etc., as the case may be. For example, an individual, subject, or patient having or at risk of developing diabetes or abnormal glucose regulation or metabolism may be an individual, subject, or patient having any one or more of the following symptoms or pathological signs commonly associated with diabetes or abnormal glucose regulation or metabolism: elevated fasting or non-fasting glucose and/or insulin levels, glucose intolerance, insulin resistance, dyslipidemia, hepatic steatosis, etc.

According to some embodiments, an individual, subject, or patient having prediabetes or abnormal glucose regulation or metabolism or an individual, subject, or patient at risk of developing diabetes may be an individual having (i) a fasting or preprandial blood glucose level in a range of about 5.5 to about 7.0 mmol per liter (i.e., about 100 to about 125 mg per deciliter), or (ii) a blood glucose level in a range of about 7.8 to about 11.1 mmol per liter (i.e., about 140 to about 200 mg per deciliter) in an oral glucose tolerance test (OGTT) about two hours after ingesting a 75-gram glucose drink. According to some embodiments, an individual, subject, or patient having diabetes may be an individual having (i) a fasting or preprandial blood glucose level of about 7.0 mmol per liter or greater (i.e., about 125 mg per deciliter or greater), or (ii) a blood glucose level of about 11.1 mmol per liter or greater (i.e., about 200 mg per deciliter or greater) in an oral glucose tolerance test (OGTT) about two hours after ingesting a 75-gram glucose drink.

According to some embodiments of the present invention, an individual, subject, or patient having or at risk of developing diabetes or abnormal glucose metabolism may be an overweight or obese individual since it is known that overweight and obese individuals have an elevated risk of developing diabetes. For humans, an overweight individual may be defined as a person or patient having a body mass index (BMI) in a range of about 25 to about 30, and an obese individual may be defined as a person or patient having a body mass index (BMI) of about 30 or greater. Alternatively, according to some embodiments, an individual, subject, or patient having, or at risk of developing, diabetes or abnormal glucose metabolism may be an individual having any known mutations and/or genetic risk factors associated with an increased likelihood of developing diabetes.

According to some embodiments, an individual, subject, or patient having diabetes may be an individual having gestational diabetes related to pregnancy. The test for identifying gestational diabetes is typically performed between the 24^{th} and the 28^{th} week of pregnancy. An individual, subject, or patient having gestational diabetes may be a pregnant human individual having a blood glucose level of about 140 mg per deciliter or greater (i.e., about 7.8 mmol/L or greater) about one hour after ingesting a 50-gram glucose drink. Alternatively, for example, an individual, subject, or patient having gestational diabetes may be a pregnant human individual having (i) a blood glucose level of about 180 mg per deciliter or greater (i.e., about 10.0 mmol/L or greater) about one hour after ingesting a 100-gram glucose drink, (ii) a blood glucose level of about 155 mg per deciliter or greater (i.e., about 8.6 mmol/L or greater) about two hours after ingesting a 100-gram glucose drink, or (iii) a blood glucose level of about 140 mg per deciliter or greater (i.e., about 7.8 mmol/L or greater) about three hours after ingesting a 100-gram glucose drink.

According to embodiments of the present invention, a therapeutically effective amount of one or more neuropeptide(s) and/or one or more TRPV1 agonist(s) or activator(s) whether applied directly to the pancreas or one or more intercostal and/or subcostal nerve(s) may be an amount effective to cause or result in a desired effect or outcome. According to embodiments of the present invention, regardless of their manner or mode of administration, a therapeutically effective amount of a neuropeptide or a TRPV1 agonist whether applied directly to the pancreas or one or more intercostal and/or subcostal nerve(s) may be an amount effective to reduce or normalize any of the known clinical symptoms or pathological signs of diabetes, prediabetes, or abnormal glucose regulation or metabolism. For example, a therapeutically effective amount may be an amount effective to reduce or normalize any one or more of the following conditions: fasting blood glucose levels, insulin resistance, glucose intolerance, or fasting or non-fasting hyperinsulinemia as measured according to any assay or technique known in the art, such as simple measurement of fasting glucose or insulin levels, an oral glucose tolerance test (OGTT or GTT), an insulin tolerance test (ITT), by using a euglycemic clamp, etc.

According to embodiments of the present invention, a therapeutically effective amount of a neuropeptide or a TRPV1 agonist whether applied directly to the pancreas or one or more intercostal and/or subcostal nerve(s) may be an amount effective to increase the level or concentration of neuropeptides, such as sP, secreted by sensory afferent neurons in the pancreas. Alternatively, according to embodiments of the present invention, a therapeutically effective amount of a neuropeptide or a TRPV1 agonist may be an amount effective to increase the synthesis, level, or concentration of neuropeptides or neuropeptide mRNA, such as sP mRNA or protein, in one or more of the dorsal root ganglia (DRGs) of thoracic segments T8 through T12, which may be observed in the cell bodies of pancreatic sensory afferent neurons.

According to embodiments of the present invention, a pharmaceutical composition comprising a TRPV1 agonist or a neuropeptide or both whether applied directly to the pancreas or one or more intercostal and/or subcostal nerve(s) may be combined with a pharmaceutically acceptable carrier. Examples of pharmaceutically acceptable carriers and other suitable additives and adjuvants for pharmaceutical compositions that may be used in combination with embodiments of the present invention for administration to an individual, subject, or patient may include those known to those skilled in the pharmacological or pharmaceutical arts. As used herein, the pharmaceutically acceptable carriers may include solvents, buffers, dispersion media, oils, liposomes, nanoparticles, coatings, surfactants, antioxidants, preservatives (e.g., antibacterial agents, antifungal agents, etc.), isotonic agents, absorption delaying agents, proteins and low and medium molecular weight polypeptides, hydrophilic polymers, amino acids, carbohydrates, sugar alcohols, metal ions, salts, preservatives, stabilizers, gels, binders, excipients, fillers, diluents, solubilizers, disintegration agents, lubricants, surfactants, penetrants, chelating agents, dyes, glidants, wetting agents, bulking agents, thickening agents, etc., and combinations thereof. Examples of pharmaceutically acceptable carriers may include, for example, substances for modifying or maintaining the pH, osmolarity, viscosity, clarity, color, sterility, stability, rate of dissolution, rate of diffusion, etc. Pharmaceutical compositions of the present invention comprising a neuropeptide for administration to the pancreas may be additionally formulated to include substances that may inhibit or avoid proteolytic degradation.

According to some embodiments of present compositions, the exact formulation route or manner of administration, and dosages of a TRPV1 agonist or neuropeptide may be chosen according to the judgment of a skilled scientist, veterinarian, pharmacologist, or physician, as the case may be, in view of the characteristics and conditions of the individual, subject, or patient to be treated. For a description of pharmaceutical compositions, carriers, formulations, methods and routes of administration, etc., that may be used for embodiments of compositions of the present invention, see, for example, Remington, The Science and Practice of Pharmacy, (University of the Sciences in Philadelphia, 21st ed., Lippincott Williams & Wilkins, 2005), the contents and disclosure of which are hereby incorporated by reference.

Except insofar as any conventional pharmaceutical carrier is incompatible with embodiments of compositions of the present invention comprising a neuropeptide or a TRPV1 agonist, their potential use in pharmaceutical compositions of the present invention is contemplated. Embodiments of the pharmaceutical compositions and formulations of the present invention may utilize different types of carriers depending on whether they are to be administered in solid, semi-solid, suspension or liquid form and whether they need to be sterile for certain routes of administration including local injection, infusion, or placement.

Embodiments of pharmaceutical compositions of the present invention for local injection, placement, implantation, infusion, etc., may be formulated with a variety of aqueous or non-aqueous solutions, suspensions, emulsions, etc. as described above, such as physiologically compatible buffers including Hank's solution, Ringer's solution, physiological saline buffer, etc. Embodiments of pharmaceutical compositions for local injection, placement, or implantation may also comprise biocompatible materials or polymers providing sustained release or restricted or locally induced diffusion as described above. As with pharmaceutical compositions for parenteral administration, solutions and suspensions for local or topical administration may be freshly prepared or resuspended from a dry preparation of a neuropeptide or a TRPV1 agonist, such as a lyophilized or spray dried preparation, prior to its use. However, embodiments of pharmaceutical compositions for local injection, placement, implantation, infusion, etc., may also be formulated as a dry or solid preparation, such as a powders, granules, etc., that may be applied directly to a desired site of action. Embodiments of pharmaceutical compositions of the present invention may be administered in a variety of unit dosage forms depending on the method of administration. For example, dosage forms may include elixirs, syrups, suspensions, sprays, gels, lotions, creams, slurries, foams, jellies, ointments, salves, solutions, suspensions, tinctures, emulsions, or any other formulation or form that is suitable for parenteral administration to the pancreas or local injection, placement, implantation, infusion, etc., at or near the site of one or more intercostal and/or subcostal nerves.

Embodiment of pharmaceutical compositions comprising a neuropeptide or a TRPV1 agonist for parenteral administration, such as intra-arterial (i.a.) injection or infusion into the pancreas, may be formulated as solutions, emulsions, suspensions, or other liquids, such as saline, dextrose solution, glycerol, and the like, which may be sterile and/or isotonic. However, a suitable carrier for parenteral administration may include aqueous or non-aqueous (e.g., oily) solvents or liposomes. Suitable formulations for parenteral administration may be in unit-dose or multi-dose sealed containers, such as ampoules, vials, bags, etc. A neuropeptide or a TRPV1 agonist of the present invention may be administered by continuous infusion or release (e.g., minipumps, osmotic pumps, etc.), single bolus, or slow-release depot formulations, etc. Solutions and suspensions for parenteral administration may be freshly prepared or resuspended from a dry preparation of a neuropeptide or a TRPV1 agonist, such as a lyophilized or spray dried preparation, prior to its use.

For topical administration, embodiments of pharmaceutical compositions of the present invention comprising a TRPV1 agonist may be formulated as a liquid or semi-solid material, such as a gel, paste, putty, ointment, cream, emulsion, patch, etc. as well as other biocompatible materials or polymers. However, embodiments of pharmaceutical compositions for topical administration may also be formulated as a dry or solid preparation, such as a powders, granules, etc., that may be applied directly to a desired site of action.

Embodiments of compositions of the present invention may be formulated so as to provide rapid, sustained, or delayed release of a neuropeptide or a TRPV1 agonist by embedding or soaking the neuropeptide or TRPV1 agonist in a matrix or network of polymeric material according to methods known in the art. Embodiments of these compositions may be formulated to restrict diffusion of a neuropeptide or TRPV1 agonist away from a location where the composition is intentionally administered or applied, such as within the pancreas or the site(s) one or more intercostal and/or subcostal nerves, which may occur by diffusion, erosion, or degradation of the network or matrix. For local administration of embodiments of compositions of the present invention, an advantage of providing sustained or restricted release is that a localized efficacious concentration of a neuropeptide or TRPV1 agonist may be achieved at a site of administration with relatively less amounts and fewer applications or injections required. Such a restricted or sustained release composition may provide targeted delivery of a neuropeptide or TRPV1 agonist while minimizing undesired side effects that may result if the neuropeptide or TRPV1 agonist diffused away from the site of administration.

Embodiments of compositions of the present invention comprising a TRPV1 agonist may be formulated to provide sustained or restricted release or diffusion of the TRPV1 agonist away from the site of its injection, application, administration, etc. Such formulations may include a variety of biocompatible materials or polymers, such as poly(2-hydroxyethyl methacrylate), ethylene vinyl acetate or poly-D-(−)-3-hydroxybutyric acid, polylactides, polyglycolides, polylactide co-glycolide, polyanhydrides, poly(ortho)esters, polypeptides, hyaluronic acid, hydrogels, collagen, fibrin, fibrinogen, fibronectin, alginate, chondroitin sulfate, carboxylic acids, fatty acids, phospholipids, polysaccharides, polynucleotides, polyvinyl propylene, polyvinylpyrrolidone, sulfated proteoglycans, dextrins, poloxamers, silicone, methylcellulose, and the like. Such compositions may comprise a semi-permeable polymer matrix or network, such as a gel, paste, putty, etc. According to some embodiments, compositions comprising a TRPV1 agonist may be molded or formed into a desired shape, such as for placement or to fill a space adjacent to an intercostal or subcostal nerve in the body of an individual, subject, or patient to promote their stimulation or activation.

According to embodiments of the present invention, a neuropeptide or a TRPV1 agonist may be administered to the pancreas of an individual, subject or patient having or at risk of developing diabetes or abnormal glucose metabolism. According to some embodiments, a composition comprising a neuropeptide or a TRPV1 agonist may be administered parenterally to the pancreas of the individual, subject or patient, such as by intra-arterial (i.a.) injection or infusion into the pancreas. Such a composition comprising a neuropeptide or a TRPV1 agonist may be administered to the pancreas singly or as part of a dosage regimen.

According to embodiments of the present invention, a TRPV1 agonist may be administered to one or more intercostal and/or subcostal nerves of spinal nerves derived from thoracic segments T8 through T12 (i.e., T8, T9, T10, T11, and/or T12) of an individual, subject, or patient having or at risk of developing diabetes or abnormal glucose metabolism. The TRPV1 agonist may be administered as part of a pharmaceutical composition in combination with a pharmaceutically acceptable carrier. According to some embodiments, a pharmaceutical composition comprising a TRPV1 agonist in combination with a pharmaceutically acceptable carrier may generally be administered to one or more intercostal and/or subcostal nerves by local injection at or near the site of one or more of these nerves. According to some embodiments, a pharmaceutical composition comprising a TRPV1 agonist in combination with a pharmaceutically acceptable carrier may conceivably be administered topically on the surface of the body of an individual above or near the location of one or more intercostal and/or subcostal nerves underneath the surface of the skin. Alternatively, according to some embodiments, a pharmaceutical composition comprising a TRPV1 agonist in combination with a pharmaceutically acceptable carrier may be administered at or near one or more intercostal and/or subcostal nerves of an individual by local placement, catheter delivery, or implantation at or near the site of one or more of these nerves, which may be achieved following access to one or more of these nerves or their surrounding tissue by surgical incision.

The translation of neuropeptide and TRPV1 agonist dosages established in mice, to mammals including human patients having or at risk of developing diabetes or abnormal glucose metabolism is known in the art. See, e.g. Hunter, R. P. et al., “Concepts and issues with interspecies scaling in zoological pharmacology,” J Zoo Wildl Med 39:517-526 (2008); Bilkei-Gorzo, A. et al. “Mutagenesis and knockout models: NK1 and substance P,” Handb Exp Pharmacol, p. 143-162 (2005); Clive, S. et al., “Forearm blood flow and local responses to peptide vasodilators: a novel pharmacodynamic measure in the phase I trial of antagonist G, a neuropeptide growth factor antagonist,” Clin Cancer Res 7:3071-3078 (2001), the entire contents and disclosures of which are hereby incorporated by reference. Effective dosing depends on the target tissue, delivery choice, and manner of formulation as may be determined in phase I clinical trials. The extremely short in vivo half-life of neuropeptides, such as sP and CGRP, (e.g. may be as little as seconds) allows single path receptor occupation in a given tissue, with extremely little if any systemic effects. For example, dosages in the nano-micromolar range have been previously applied in humans with no adverse effects.

Having described several of the embodiments of the present invention in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

### EXAMPLES

The above observations show that primary sensory afferent TRPV1+ neurons in the pancreas may play an important role in diabetes progression through the local release of neuropeptides including substance P (sP) as a prototype molecule. These neurons appear to play a role in establishing insulin homeostasis, which becomes imbalanced with hypofunctional TRPV1 function and/or lowered amounts of sP release, thus leading to β-cell stress and diabetes progression. Although pancreatic injection of neuropeptides or stimulants of TRPV1+ neurons may provide therapeutic benefit, the need for repeated pancreatic injections may make such approaches challenging. Instead, it is proposed that stimulation of pancreatic TRPV1+ sensory neurons to treat diabetes may be achieved by stimulating or activating these neurons remotely through surgical, mechanical, electrical, and/or chemical techniques. For example, stimulation of more accessible neurons derived from the same dorsal root ganglions (DRGs) as pancreatic TRPV1+ sensory neurons (i.e., having cell bodies in the same DRGs as pancreatic TRPV1+ sensory neurons) may relay the stimulation signal to these pancreatic TRPV1+ sensory neurons to cause local release of neuropeptides in the pancreas.

### Example 1

### Unilateral Axotomy of Thoracic Spinal Nerves in Mice

Pancreatic islets and associated lymph nodes are highly innervated by primary sensory afferent neurons, with cell bodies in spinal dorsal root ganglia. See, e.g., Lindsay, R. M. et al., “Spinal cord contains neurotrophic activity for spinal nerve sensory neurons. Late developmental appearance of a survival factor distinct from nerve growth factor,” Neuroscience 12:45-51 (1984), the entire contents and disclosure of which is hereby incorporated by reference. These nerves contain diverse neuron subclasses, prominently including nociceptors that express the cation channel, TRPV1. Activation of TRPV1 triggers Ca²⁺ flux and release of bioactive neuropeptides as a local efferent function, which may impact local autoimmune reactions and diabetes progression as described above.

Nerve injury models have increased knowledge of the contributions of sensory nerves in the control of immune responses. See, e.g. Araki, T. et al., “Identification of genes induced in peripheral nerve after injury: Expression profiling and novel gene discovery,” J Biol Chem 26:26 (2001). A preferred peripheral nerve injury model is compression or partial axotomy of the sciatic nerve. See, e.g., Chao, T. et al., “Chronic nerve compression injury induces a phenotypic switch of neurons within the dorsal root ganglia,” J Comp Neurol 506:180-193 (2008), the entire contents and disclosure of which is hereby incorporated by reference. However, the peripheral nerve injury response and associated chronic pain remains incompletely understood. Recently, it has been observed that numerous changes in gene expression are observed following various forms of peripheral nerve injury. These changes involve the ipsilateral DRG, and prominently include capsaicin-sensitive (i.e., TRPV1 positive) sensory afferent neurons with upregulation of the associated neuropeptides. See, e.g. Jansco, G. et al., “Peripheral nerve lesion-induced uptake and transport of choleragenoid by capsaicin-sensitive c-fibre spinal ganglion neurons,” Acta Biol Hung 53:77-84 (2002), the entire contents and disclosure of which is hereby incorporated by reference.

Upregulation of neuropeptide expression, even in neurons previously negative for expression of these genes might provide a physiological strategy to improve the neuropeptide deficiency underlying Diabetes pathoetiology. There is evidence that a post-injury response is translated or communicated to the spinal column and includes sP elevation. See, e.g. Neumann, S. et al., “Inflammatory pain hypersensitivity mediated by phenotypic switch in myelinated primary sensory neurons,” Nature 384:360-364 (1996), the entire contents and disclosure of which is hereby incorporated by reference. Therefore, it is determined whether following ipsilateral axotomy of one or more sensory neurons, contralateral DRGs whose axonal connections to the pancreas remain unimpaired, might respond to axotomy of contralateral neurons with improved sP release and supply to the pancreas. As described below, unilateral axotomy, but generally not bilateral axotomy (which may have much less of an effect), downstream of T8 through T12 DRGs, rapidly generates a broad neuropeptide overexpression in previously sP-negative DRG neurons resulting in enhanced pancreatic neuropeptide levels, which protect from islet inflammation, and normalized insulin resistance with acute reversal of overt diabetes in mice for a period of weeks to months.

### Materials and Methods

### Animals.

NOD/LtJ, NOD.scid, and BDC2.5 TCR transgenic NOD mice (BDC2.5-NOD) mice are purchased from Jackson laboratories and maintained in a vivarium under approved protocols (female type 1 diabetes (T1D) incidence 85-90%). Animal handling procedures are as described. See, e.g., Razavi et al. (2006), supra. For axotomy, a 0.5 cm shallow incision is followed by spinal nerve track exposure using 30½ gauge needles. Spinal nerves are then looped and pulled, and about 4-5 cm of the nerve bundle is removed. Severed nerves are folded in opposite directions to result in initially about 1 cm or greater gap between the severed tracks. Muscle tissue is replaced in its natural position, and mice are sutured and rested under mild analgesic cover. Lumbar nerve surgery employs similar technique. Mock surgeries omit the nerve cut.

### Immunofluorescence and Di-I Anterograde Tracing of Pancreatic Innervations.

For dye tracing experiments, spinal nerves are exposed adjacent to the corresponding dorsal root ganglia, and the lipophilic tracer Di-I (Molecular Probes, Eugene Oreg.) is applied. Mice are then rested for four days, and tissue is cryopreserved for subsequent immunohistochemistry. For immunofluorescence, frozen pancreas sections are fixed in 2% paraformaldehyde and blocked with 5% normal donkey serum (Jackson) diluted in TRIS-buffered saline. Sections are stained with polyclonal antibodies against GFAP (Signet Pathology Systems, Dedham, Mass.) and guinea-pig antibody against insulin (DAKO, Carpinteria, Calif.). Bound antibodies are detected with FITC-conjugated donkey anti-rabbit and biotinylated donkey anti-guinea pig IgG (Jackson) with Streptavidin Alexafluor 633 (Molecular Probes). TRPV1 and sP staining is performed on snap-frozen dorsal root ganglia sections using a rabbit polyclonal to TRPV1 (Oncogene) and rat monoclonal to sP (Calbiochem). Bound antibodies are detected with FITC-conjugated donkey anti-rabbit and biotin-conjugated donkey anti-rat (Jackson) followed by streptavidin alexa-546 (Jackson). When biotinylated antibodies are used, sections are blocked with an avidin/biotin blocking kit (Vector). Slides are analyzed by confocal microscopy.

### Histology and Scoring of Insulitis.

Pancreata are fixed in 10% buffered formalin. Histological sections are stained with hematoxylin and eosin at six levels through the pancreas. To assess insulitis severity in the pancreas, three blinded observers scored at the following scale: 0=normal islet; 1=peri-insulitis or encroachment of <25% of the islet surface area; 2=invasive infiltration of 25-50% of the islet surface area; 3=invasive infiltration of >50% of the islet surface area.

### BDC2.5 T Cell Purification, Labeling, Transfer, FACS.

NOD.Cg-Tg(TcraBDC2.5)1Doi Tg(TcrbBDC2.5)2Doi/DoiJ (BDC2.5) mice (The Jackson Laboratory) are used to assess the proliferative capacity of T cells in the lymph nodes of mice with thoracic nerve transection. Spleens from BDC2.5 mice aged between 8-10 weeks of age are harvested and placed in AIM-V (Gibco) containing 5% FBS and dispersed. The resultant suspension is filtered through a 75 um cell strainer into a 50 mL Falcon tube. CD4+ T cells are purified using EasySep positive selection using a Mouse CD4 Selection Cocktail and magnetic bead purification (Stem Cell Technologies) according to manufacturers suggestions. Following purification, cells are then labeled with Vybrant CFDA SE Cell Tracer Kit (Molecular Probes) per manufacturer's suggestions using a 2 uM working solution of CFDA SE. Immediately after labeling, about 2-5×10⁶ cells are injected intravenously (i.v.), and mice are rested for about 96 hours. Pancreatic and axillary lymph nodes are harvested, stained with biotinylated anti-Vβ4 with APC-conjugated Streptavidin, and analyzed by FACS analysis.

### Flow Cytometry

Splenocytes are stained with FITC, PE, and APC conjugated antibodies to CD3, CD4, CD8, CD25, Foxp3, and B220 (BD Pharmingen, not all combinations are shown). Live events are collected based on forward-scatter and side-scatter profiles on a FACScan flow cytometer (BD) and analyzed using FlowJo software (Stanford University).

Adoptive Transfer of Splenocytes to NOD.scid Mice.

In adoptive transfer experiments, 15×10⁶ pooled splenocytes per mouse from 5-8 diabetic NOD females receiving axotomy (AX) or mock surgery are injected intravenously (i.v.) into irradiated (300 rad) 6 to 8 week old NOD.scid recipients.

### Substance P RT-PCR and ELISA.

Surgery and mock surgery are performed as described, and mice are rested for two weeks. Contralateral thoracic T9-T12 DRGs are harvested and pooled (20 mice/group) for RNA purification using Trizol reagent (Invitrogen). RNA is reverse transcribed, and PCR is performed using primers specific for Substance P. Forward: 5′-ATGAAAATCCTCGTGGCGGT-3′, Reverse: 5′-CAGCATCCCGTTGCCCATT-3′. β-actin is used as a loading control for PCR (Ambion).

Tissue levels of sP are accessed via sP Correlate-EIA kit (Assay Designs). Tissue samples are boiled for about 1 hour in 2M acetic acid and centrifuged 15 min at 16,000 g, and supernatants are evaporated in a speed-vap (Hetovac, Scandinavia) overnight at room temperature. Peptides are then reconstituted in ELISA sample buffer and sP levels are determined as per manufacturer's instructions.

### Statistics

Numeric outcomes are analyzed with two-sided Mann-Whitney or Walsh tests, and incidence data are analyzed by life tables and Fishers exact test. Significance is set at 5%.

### Results

### Pancreas Denervation.

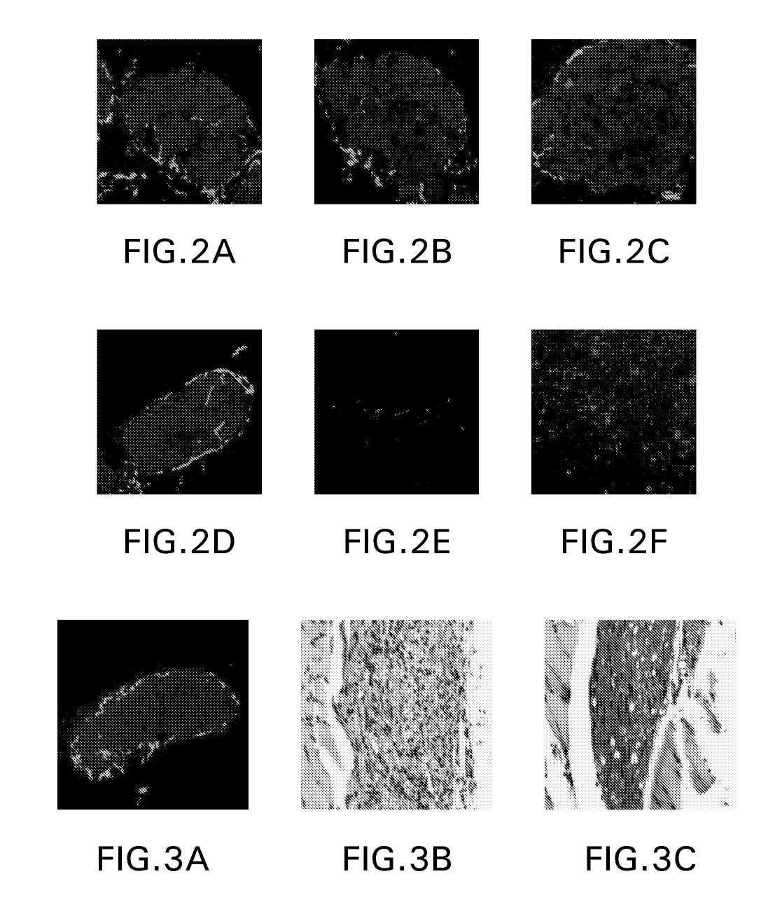

As the precise sensory innervation of mouse pancreas has not been reported, anterograde tracing with the lipophilic fluorescent dye, DiI, is used. These studies show that the bulk of NOD mouse pancreas innervation derives from DRGs at T8 through T12 (FIG. 2). Dye tracings derive about equally from left and right side DRGs, and are found both inside and outside of the islet (FIGS. 2A, 2B, and 2C). Dye tracks are also observed in exocrine pancreas and pancreatic lymph node tissue (FIGS. 2E and 2F). There is no interfering auto-fluorescence, and there is no pancreatic dye accumulation after labeling axons from other spinal nerves (FIG. 2D). Based on these findings, a surgical protocol is designed for unilateral spinal nerve axotomies at sites distal of DRGs from one or more segments T9 through T12. Axon bundles are physically extended (e.g. greater than about 2 cm) before removing about 4 cm on the left or right branches with similar results. In some experiments, bilateral axotomies are also performed (see below).

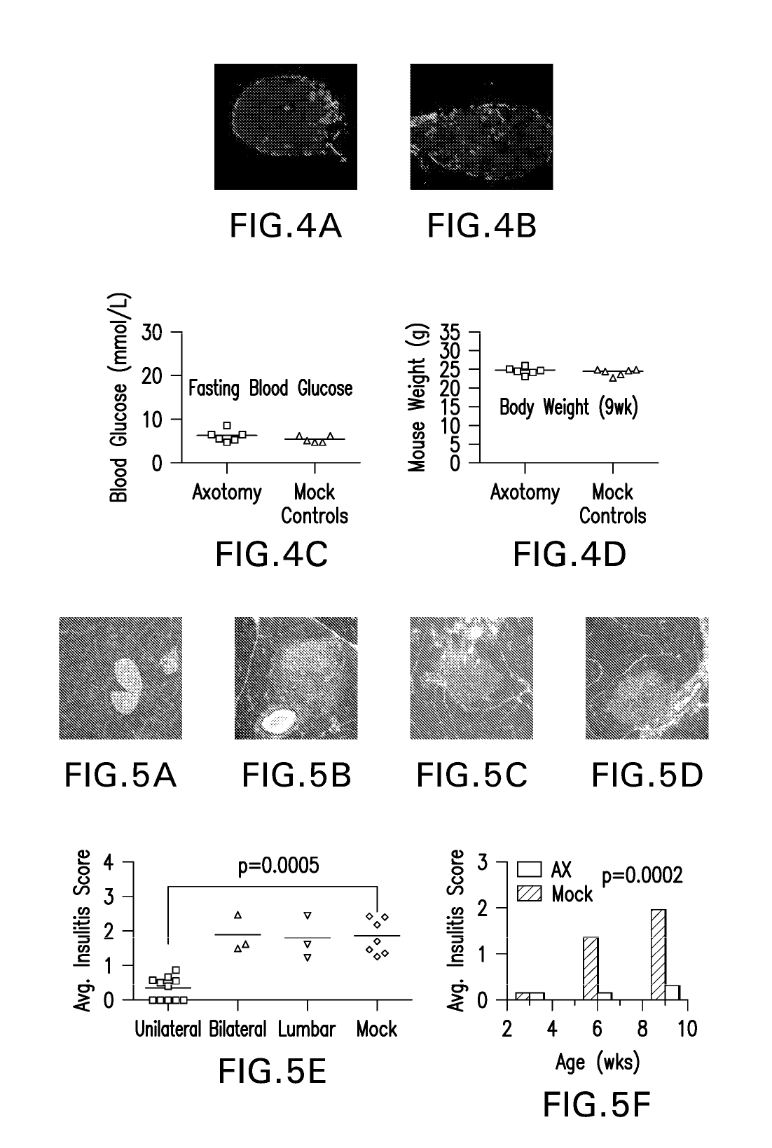

Applying DiI on the proximal of the axotomy scar 1 month post-surgery showed no dye traces in the pancreas, indicative of a failure to regenerate pancreas innervation (FIG. 3A). Histology of serially sectioned nerve scars in randomly chosen animals post-axotomy demonstrated degeneration, leukocytic infiltration, and no evidence of regeneration (FIGS. 3B and 3C). Mice tolerate the axotomy well and remain healthy. Peri-islet glia and islet structure (including α- and β-cell content), glucose metabolism, and body weights 3-5 weeks after surgery, are each indistinguishable compared to animals that underwent mock surgery (FIGS. 4A through 4D).

T9-T12 Axotomy Protects from Islet Inflammation and T1D Development.

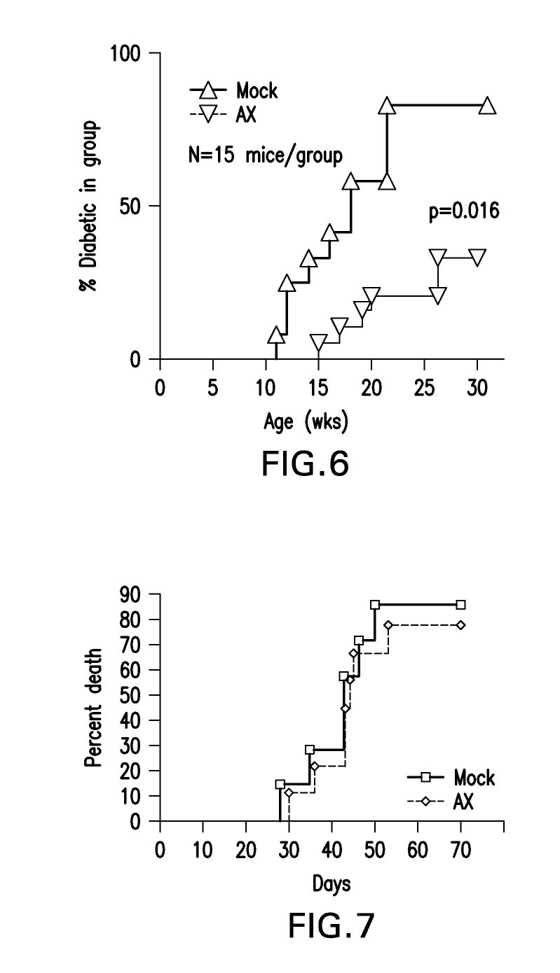

Axotomies of spinal nerves from thoracic segments T9 through T12 in 21 day old weaned NOD females are performed. This age is chosen to perhaps limit, but not prevent, priming of islet autoreactive T cell pools. The progression of NOD mouse prediabetes is characterized by a slow accumulation of lymphocytes and antigen presenting cells at the pSC barrier, starting at about 3 weeks of age. See, e.g. Atkinson, M. A. et al., “The NOD mouse model of type 1 diabetes: as good as it gets?” Nat Med 5:601-604 (1999), the entire contents and disclosure of which is hereby incorporated by reference. By about 10 weeks of age, the pSC mantle of most islets is breeched with only few pSC remaining, but minimal loss of β-cell mass. By 10 weeks of age, all but 5 mice with unilateral T9-12 axotomy have no islet inflammation, and 6 unilaterally axotomized animals have minimal insulitis (FIGS. 5A and 5E). All animals that undergo mock surgery, bilateral axotomy, or lumbar axotomy have invasive insulitis (FIGS. 5B through 5E). NOD mice receiving unilateral axotomy have decreased incidence of diabetes compared to mock-treated controls further confirming the role of sensory afferent neurons in diabetes progression (FIG. 6). Diabetes is confirmed by diabetic blood glucose measurements on 2 consecutive days (greater than 13.8 mM per liter; SureStep, Life Technologies Inc., Burnaby, British Columbia, Canada).

Bilateral axotomy of spinal nerves from the same thoracic segments as well as axotomy of non-pancreas-innervating spinal nerves are consistently ineffective. Thus, the axotomy effect is anatomically site-specific and requires unilateral damage, suggesting that the effect involves or is mediated by the undamaged contralateral axons and dorsal root ganglia. Insulitis and diabetes protection is neither complete nor permanent as severe insulitis developed in many axotomized animals after the age of 35 weeks, although the delay in insulitis onset and progression was significant (p=0.0002) (FIGS. 5F and 6). The delay in islet inflammation is reflected in the delay of clinical diabetes onset and a significant reduction of disease incidence (p=0.016) (FIG. 6). Interestingly, despite clear disease protection in 10-12 week old axotomized mice, splenocytes from these animals are perfectly able to transfer disease to NOD.scid recipients (FIG. 7), thus mapping lymphocyte access to the pancreas in NOD mice to sensory afferent neurons innervating the pancreas and pancreatic lymph nodes.

### Reversal of Overt T1D-Like Disease in NOD Mice by Unilateral T9-T12 Axotomy.

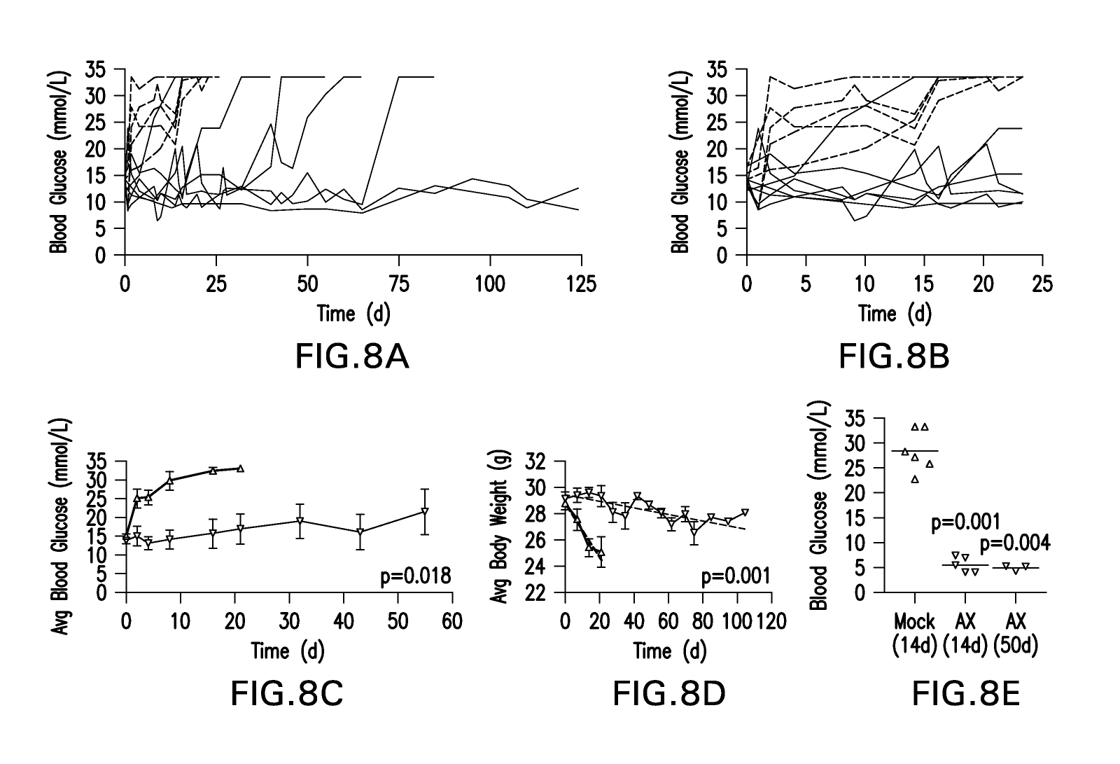

The response of newly diabetic NOD females to unilateral axotomy is also determined. Axotomies are performed similarly to the above, but in mice that already have diabetic hyperglycemia (e.g., about 15 mmol/L) on two consecutive days. Individual mice are then monitored daily for blood glucose levels (FIGS. 8A and 8B) and body weights (FIG. 8D) without insulin treatment. Average glucose levels are also determined (FIG. 8C). As expected, mice with mock surgery (broken lines) have the typical progression to severe hyperglycemia concomitantly with loss of body mass and requirement of euthanasia usually within days of onset. This abrupt course is typical for mice and differs from diabetic human patients having a period of weeks to months (i.e., a so-called “honeymoon period”) with little or no insulin therapy required, which may reflect the relatively smaller absolute β-cell mass present in rodents with little room for functional reserves.

Axotomy reduced hyperglycemia overnight reaching high normal, but tolerable, glucose levels within days (FIG. 8B). There are considerable daily swings in glycemia, but fasting glucose levels are near normal at 14 and 50 days after onset in surviving axotomized mice, suggesting that the reduced β-cell mass at diabetes onset is suboptimal for normal metabolism but sufficient for survival (FIG. 8E). The first animal reverted to diabetes 15 days after axotomy, but survived several weeks. The longest surviving individual mouse survived for more than 6 months post-axotomy. When individual courses are averaged (FIG. 8C, mean blood glucose ±SD), differences between animals receiving mock surgery or axotomy are significant (p=0.018), and axotomized mice sustained metabolic control and body mass much better than after mock surgery (FIGS. 8D and 8E). These animals received no insulin therapy, and the variability observed among axotomized mice is believed to be the result of residual β-cell mass at the time of onset, which may be compounded by differences in β-cell regeneration. Perhaps adjunct post-axotomy therapies, such as slow-release insulin therapy, may be optimized to stimulate β-cell regeneration or neuronal signaling patterns.

Mechanism of Insulitis and Disease Protection with Unilateral T9-T12 Axotomy.

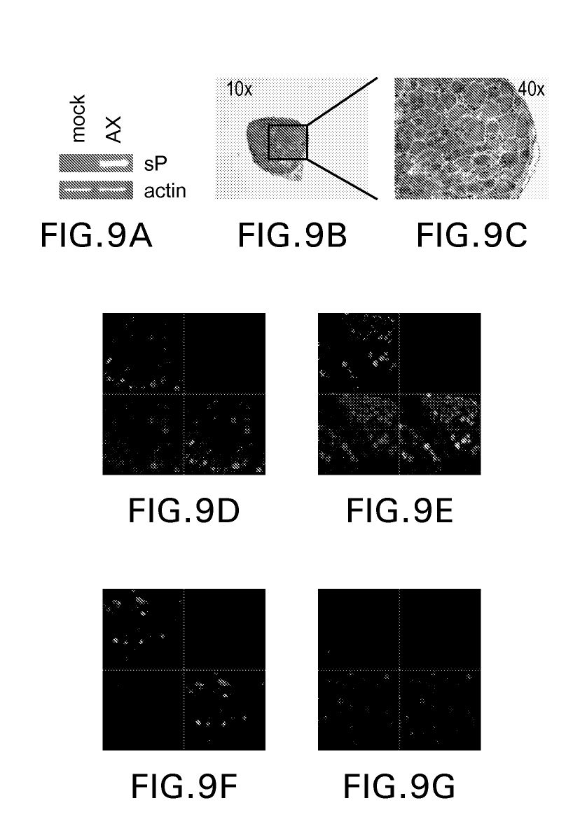

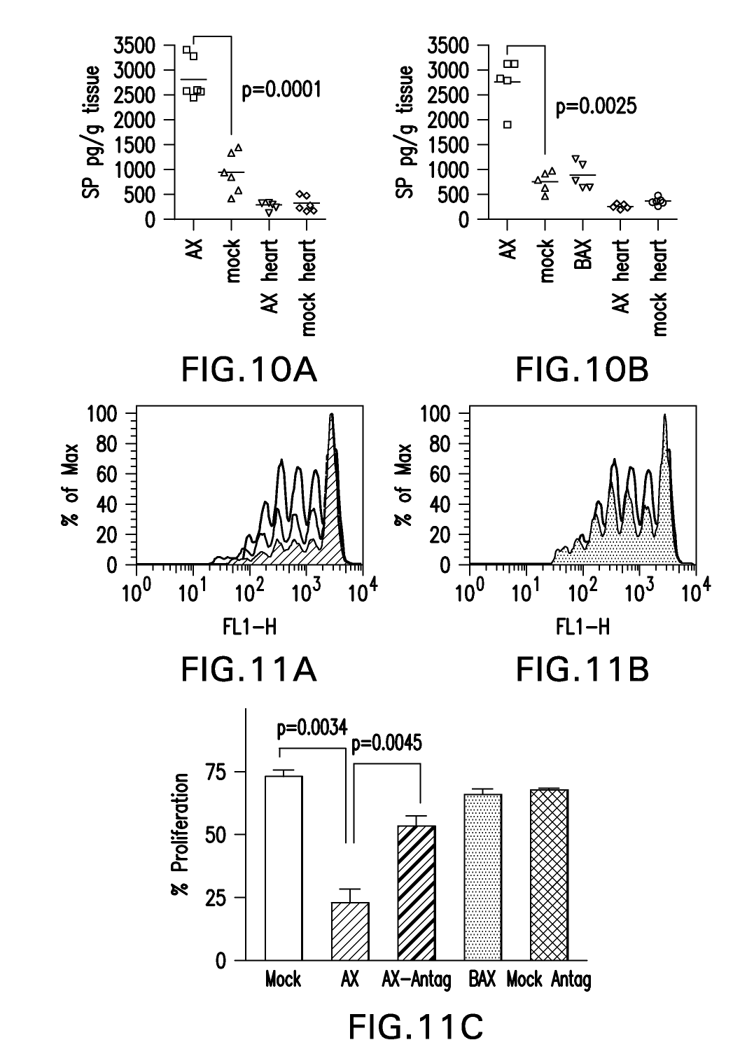

The rationale for the studies in the present example is to determine if a functional strategy can be devised to promote an increase in endogenous sP expression in the pancreas and pancreatic lymph nodes. In response to T9-T12 unilateral axotomy, there is a dramatic enhancement of sP message in contralateral DRGs within 1-2 days, indicative of de novo gene induction (FIG. 9A). Dorsal root ganglia originating from T9-T12 regions are also analyzed by histology and immunofluorescence (FIGS. 9B through 9G). In control DRGs, sP is expressed in a subset of neurons that also express TRPV1 (FIG. 9D). However, after contralateral axotomy, a large proportion of neurons, most of which are negative for TRPV1 expression before the procedure, stain positively for sP post-axotomy (FIG. 9E). sP-deficient and TRPV1-deficient animals are used as staining controls (FIGS. 9F and 9G). This remarkable contralateral phenotypic switch demonstrates that nerve injury not only induces profound responses in the affected segment and dorsal horn, but also rapidly involves the contralateral segment. While the mechanism remains unclear, it could be in part central and in part spinal. It is also unclear how sP release from TRPV1-negative neurons is controlled, and if sP release in these circumstance may be linked to Ca²⁺ flux or other ionic events. At any rate, enhanced sP expression does impact pancreatic sP concentrations as measured by ELISA of pancreas tissue extracts. Elevated pancreatic sP concentrations are found after unilateral axotomy, but not in bilaterally axotomized mice or mock-treated controls, for prolonged periods of time. Elevation of sP following unilateral axotomy of nerves from T9-T12 segments is pancreas-selective and not observed in the heart at different time points (FIGS. 10A and 10B).

These observations demonstrate that surgical nerve injury generates de novo expression of sP in previously sP-negative and TRPV1-negative contralateral DRG neurons that innervate the pancreatic region with a lack of discernable effects following bilateral axotomy. It remains unclear if and how both dorsal horns in a given thoracic segment communicate contralaterally. Sensing the loss of post-axotomy innervation and/or neuropeptide release in the terminal region may each contribute to the response. Ligation of insulin receptors, which are abundant on TRPV1+ afferent neuron terminals, lowers the activation threshold of these neurons. In the insulin-rich pancreatic islet milieu, this generates a tonic local neuropeptide release. Such ligation may also generate tonic electrical afferent signals to the spine and CNS.

The disease protection by T9-T12 axotomy observed in mice is transient. However, these time periods may suggest considerable time periods of protection in human diabetics and prediabetics (e.g., several years) if this technology can be translated to humans. The observed phenotypic switch in sP expression is also time limited. The sparse literature suggests 1-2 weeks but only ipsilateral effects have been measured. Given the limited β-cell mass in rodents, several factors may determine the life time of disease protection by axotomy. However, axotomy may be staggered (e.g., T9 & T11 followed by T10 & T12) to allow for repetition of the axotomy procedure if the effectiveness of the previous treatment wanes.

### sP is Critical for Disease Protection by Axotomy.

The rapidity of diabetes reversal is impressive with rapid and effective normalization of pathological phenotypes. To determine the molecular mechanism of this process, the effect of sP antagonists is analyzed. See, e.g., Rupniak, N. M. et al., “P-Glycoprotein efflux reduces the brain concentration of the substance P (NK1 receptor) antagonists SR140333 and GR205171: a comparative study using mdr1a−/− and mdr1a+/+mice,” Behav Pharmacol 14:457-463 (2003), the entire contents and disclosure of which are hereby incorporated by reference.

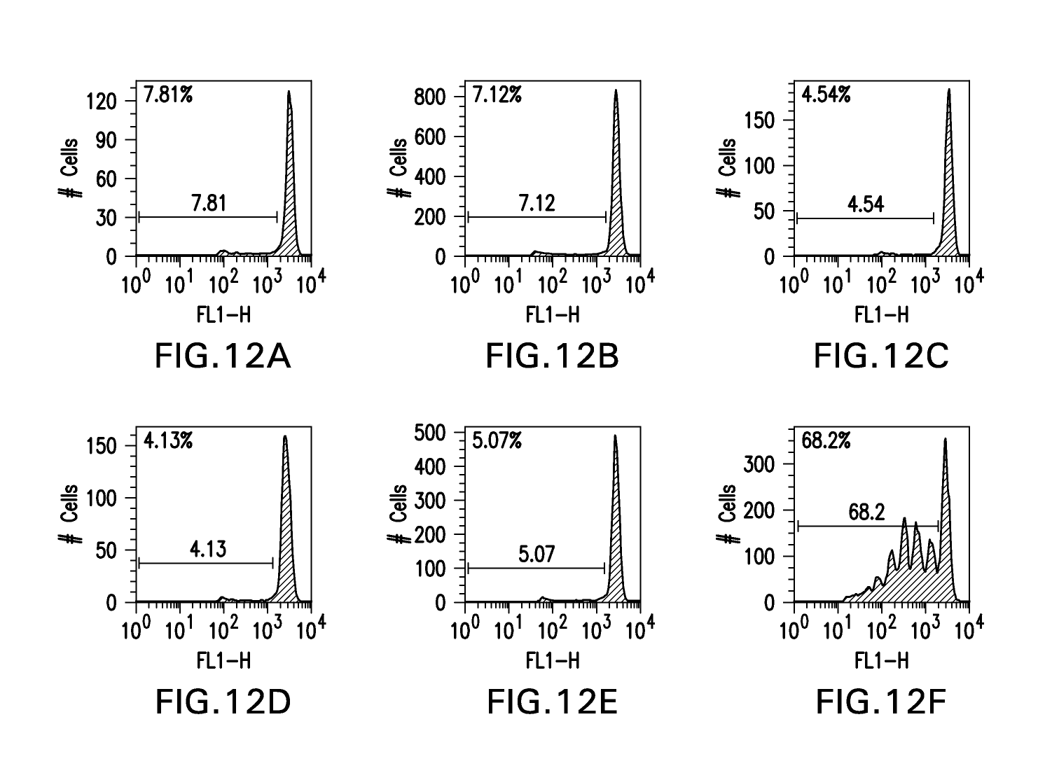

Homogeneous T cell receptor transgenic BDC2.5 T lymphocytes labeled with a fluorescent dye CFSE are adoptively transferred to measure in vivo proliferative expansion (FIG. 10 and FIG. 11). BDC2.5 T cells migrate to the pancreas and pancreatic lymph node tissue within 24-48 hours. Following axotomy, most islets remain T cell free, with minor infiltrations in only a few. BDC2.5 T lymphocytes accumulate in draining pancreatic (but not axillary) lymph nodes, and about 70% immediately divide (FIG. 11A, thick line). However, proliferation is greatly reduced following axotomy (FIG. 11A, shaded area). This effect critically requires sP since in vivo treatment with the NK1R (sP receptor) antagonist, N-Acetyl-L-tryptophan 3,5-bis(trifluoromethyl)benzyl ester significantly increased the number of proliferating cells in axotomized mice (p=0.0045) (FIG. 11A, thin line, and summarized in FIG. 11C). BDC2.5 proliferation following bilateral axotomies (FIG. 11B, shaded area) is not significantly different from that in mock surgery mice (thick line), and mock surgery mice treated with the NK1R antagonist do not show significantly altered proliferative capacity (FIGS. 11C and 12F). Axillary lymph nodes are examined in all mice as a control for basal proliferation, and no BDC2.5 expansion is noted (FIGS. 12A through 12E).

These observations establish that the induction of sP production in TRPV1-negative sensory neurons represents a considerable tissue-selective nerve injury response in contralateral DRGs sufficient to normalize the diabetes-associated pathological phenotypes. The protection from diabetic autoimmunity also critically involves NK1R binding. NK1R is abundantly expressed on recently activated islet infiltrating T lymphocytes, and its ligation may lead to T cell death via a non-classical, nur77-dependent programmed death mechanism. See, e.g. Persson-Sjogren, S. et al. (2005), supra; Persson-Sjogren, S. et al., “Remodeling of the innervation of pancreatic islets accompanies insulitis preceding onset of diabetes in the NOD mouse,” J Neuroimmunol 158:128-137 (2005); Castro-Obregon, S. et al., “A ligand-receptor pair that triggers a non-apoptotic form of programmed cell death,” Cell Death Differ 9:807-817 (2002); Castro-Obregon, S. et al., “Alternative, nonapoptotic programmed cell death: mediation by arrestin 2, ERK2, and Nur77,” J Biol Chem 279:17543-17553 (2004), the entire contents and disclosures of which are hereby incorporated by reference. Thus, unilateral T9-T12 axotomy constitutes a therapy for diabetes that re-establishes physiological minute-to-minute glucose control.

Collectively, these observations delineate a fundamental role for sP in protecting pancreatic islets from metabolic stress associated with rising insulin resistance, hyperinsulinism, and autoimmune attack via a strictly local neurogenic mechanism involving endogenous pancreatic sP levels, which may be elevated by axotomy to combat disease. An analogous axotomy protocol involving thoracic sympathetic nerve bundles is a commonly used procedure in pediatric and adult patients with hyperhydrosis. These examples support translational efforts in treating diabetic and pre-diabetic humans.

### Example 2

### Unilateral Stimulation of Thoracic Spinal Nerves with TRPV1 Agonists in Mice

In this example, unilateral activation of sensory nerve strands derived from T8-T12 spinal thoracic segments with a chemical compound known to be an agonist, activator, ligand, etc., of TRPV1, such as capsaicinoid compounds including capsaicin, etc., and other capsaicin analogs, such as the high-affinity resiniferatoxin (RTX). See, e.g. Almasi, R. et al., “Effect of resiniferatoxin on the noxious heat threshold temperature in the rat: a novel heat allodynia model sensitive to analgesics,” Br J Pharmacol 139:49-58 (2003), the entire contents and disclosure of which is hereby incorporated by reference. In contrast to axotomy or other surgical techniques, this strategy may be more TRPV1-specific since axotomy also affects sympathetic fibers.

### Materials and Methods

Mice. NOD/LtJ, NOD.scid, and BDC2.5 TCR transgenic NOD mice (BDC2.5-NOD) mice are purchased from Jackson laboratories and maintained in a vivarium under approved protocols (female type 1 diabetes (T1D) incidence 85-90%). Animal handling procedures are as described previously. For RTX treatment, a 0.5 cm shallow incision is made to expose the spinal nerve track to be treated. About 10 nmol RTX in about 1 microliter is then applied to the spinal nerves. After 10 minutes, the excess is removed. Muscle tissue is replaced in its natural position, and mice are sutured and rested under mild analgesic cover. Lumbar nerve surgery employs similar technique. Mock surgeries omit the RTX application.

Plasma extravasation in kidney and pancreas. About 1 mg of Evans Blue dye in PBS is injected intravenous (i.v.), and relevant tissue samples are acid extracted for spectrometric quantitation of the blue dye.

Glucose tolerance test (GTT) and insulin tolerance test (ITT). For GTT, mice fasted for 16 hours received 0.75-1.0 g glucose/kg body weight is injected and relevant measurements are made. For ITT, mice received 0.75 U insulin/kg body weight of human regular insulin (Eli Lilly) and relevant measurements are made. However, 2 U insulin/kg body weight is used in ob/ob mice.

Flow cytometry. T cells are stained with antibodies to CD4, Foxp3, and NK1R and subjected to flow cytometry to determine CD4+, Foxp3+ regulatory T cells displaying the NK1 receptor following treatment with a TRPV1 agonist (RTX).

### Results

### T8-T12 Local TRPV1 Agonist Application.

The thoracic spinal nerves T8 through T12 exiting the spinal column split into thoracic intercostal nerves and nerve fibers that lead to thoracic splanchnic nerves via ganglion of the sympathetic chain, and these thoracic splanchnic nerves include sensory afferent nerves terminating in the pancreas. Since surgical access to the splanchnic nerves is challenging and invasive, stimulation or activation of the thoracic intercostal nerves with a TRPV1 agonist is tested to determine whether this approach by itself would be sufficient to alter the contralateral DRG milieu which includes pancreatic TRPV1+ sensory afferent neurons to alter the functioning of these sensory neurons of the pancreas as with previous experiments following axotomy.

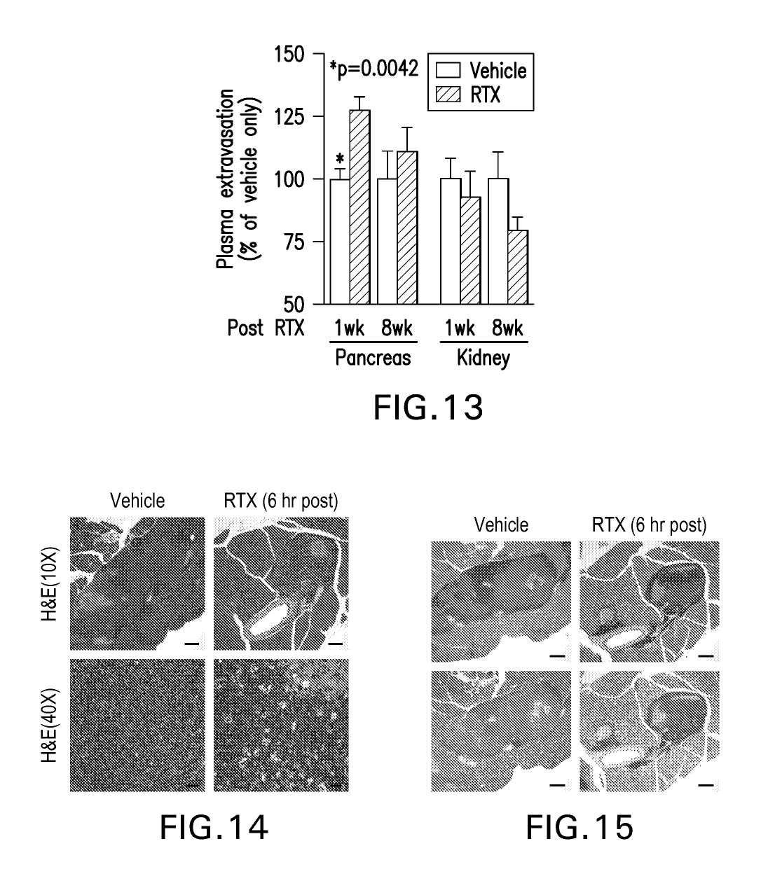

Since the exact mechanism of contralateral changes in the DRG milieu that generate sP overexpression in contralateral neurons remains unknown, it is important to confirm if local TRPV1 agonist (RTX) application directly onto the micro-surgically developed axon trunks of intercostal thoracic nerves derived from T8-T12 changes sP expression. TRPV1 agonist (RTX) application (20 nM) significantly enhances vascular leakage in the pancreas (p=0.0042) that lasted for weeks but declined by 2 months (FIG. 13). Ipsilateral and contralateral kidneys are used as a control tissue to rule out possible systemic TRPV1 agonist effects. There is no significant difference (p>0.05) between vehicle and TRPV1 agonist (RTX) application in either kidney so data are pooled. These data demonstrate that unilateral TRPV1 agonist (RTX) application on surgically exposed thoracic intercostal nerves T8-T12 (or T10-T11 only, data not shown), does induce a shift in sP expression with pancreas-selective enhancement of vascular leakage, bypassing the hypofunctional TRPV1 in the NOD mouse through expression in previously sP-negative and TRPV1-negative neurons.

### TRPV1Agonist Effects on Inflammatory Lesions.

The pancreatic rise in sP (and associated neuropeptides, such as CGRP) have dramatic effects reminiscent of the response of direct sP injection into the pancreas. Within hours following TRPV1 agonist (RTX) application, lymphocytes infiltrating the islet show lacunar regions of spreading cell death (FIG. 14). Considering the three-dimensional character of islets, this effect is rapid and dramatic. The lacunar centrifugal (i.e., “spoked wheel”) lesions imply fratricide among the infiltrating lymphocytes, consistent with transient activation following NK1R ligation by sP leading to massive local mediator/cytokine release prior to undergoing non-classical apoptosis.

To analyze the suspected cell death process in islet infiltrates following RTX application, histochemical measurements of DNA strand breaks typical of cell death pathways, but not necrosis, are employed. Positive TUNEL staining is observed in all areas of the infiltrate, but considerably more pronounced in T cell predominant CD3+ areas (FIG. 15). No TUNEL staining is observed in islets from vehicle-treated control animals. These data provide the first known demonstration of direct lymphocyte killing by local neuropeptide secretion potential from primary sensory afferent neurons. These observations are consistent with the notion that diabetes is fundamentally controlled in several respects by sensory afferent neurons, which affect the tissue homing and tissue accumulation of infiltrates, along with direct effects on the survival, stress level, and functions (e.g., measured as hyperinsulinism, rising insulin resistance, etc.) of β-cells.

These observations may have implications beyond diabetes by suggesting possibly fundamental roles for sensory afferent neurons in affecting immunocompetence. This is supported by evidence from rare patients with CIPA syndrome who lack sensory neuron function. These patients succumb early in life to progressive tissue infectious lesions that fail to attract immune attention. Thus, based on the present work, targeted nerve stimulation or activation may provide a way to treat a number of clinical conditions characterized by chronic progressive inflammation. There is a large natural variation in the sensitivity of sensory nerves, due in part perhaps to the very large haplotype diversity of the polymorphic TRPV1 gene, with low sensitivity generally predisposing to autoimmunity or other conditions associated with tissue inflammation.

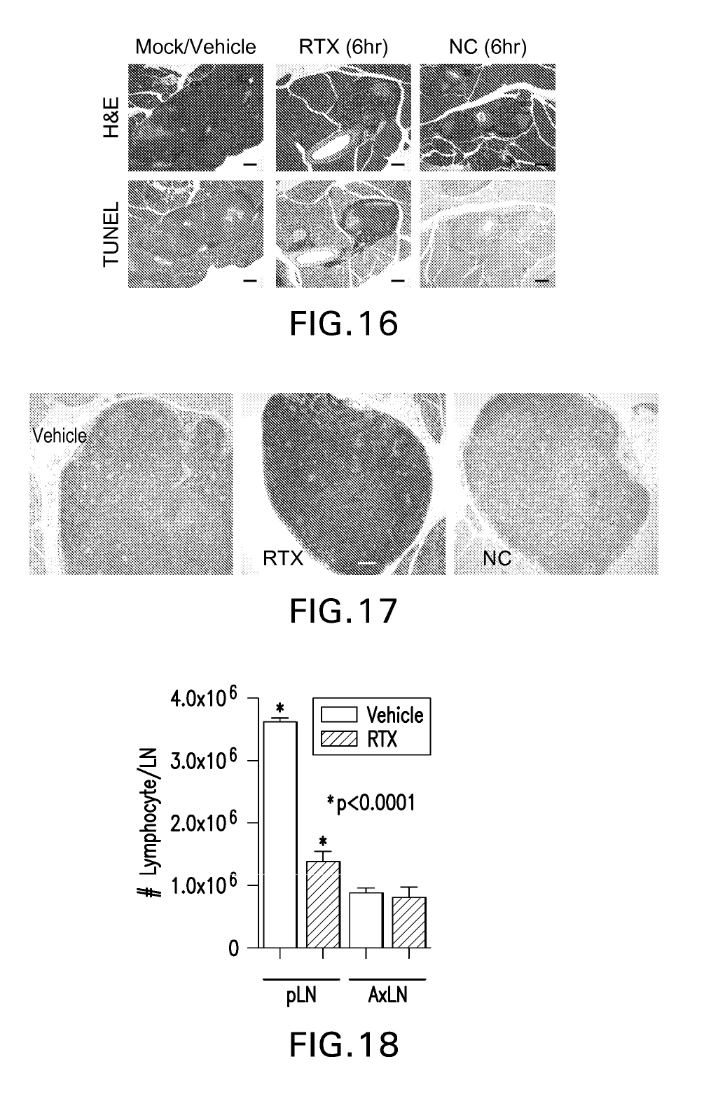

Local TRPV1 agonist application and axotomy techniques of sensory afferent thoracic intercostal wall branches are compared. Both treatments have similar effects on lymphocytic infiltrations in the islet (FIG. 16) and on lymphocyte populations in the pancreatic lymph nodes (FIG. 17), which receive their sensory innervation from the same thoracic segments. However, it is consistently observed that TRPV1 agonist (RTX) treatment has more rapid and more profound effects than axotomy, perhaps reflecting the fact that TRPV1 agonists may rapidly activate TRPV1 and immediate propagation of depolarizing signals. In contrast, the axotomy response may involve more complex events. However, it is not known if these differences are meaningful since both treatment strategies revert diabetes-associated whole animal pathology to very similar extents.

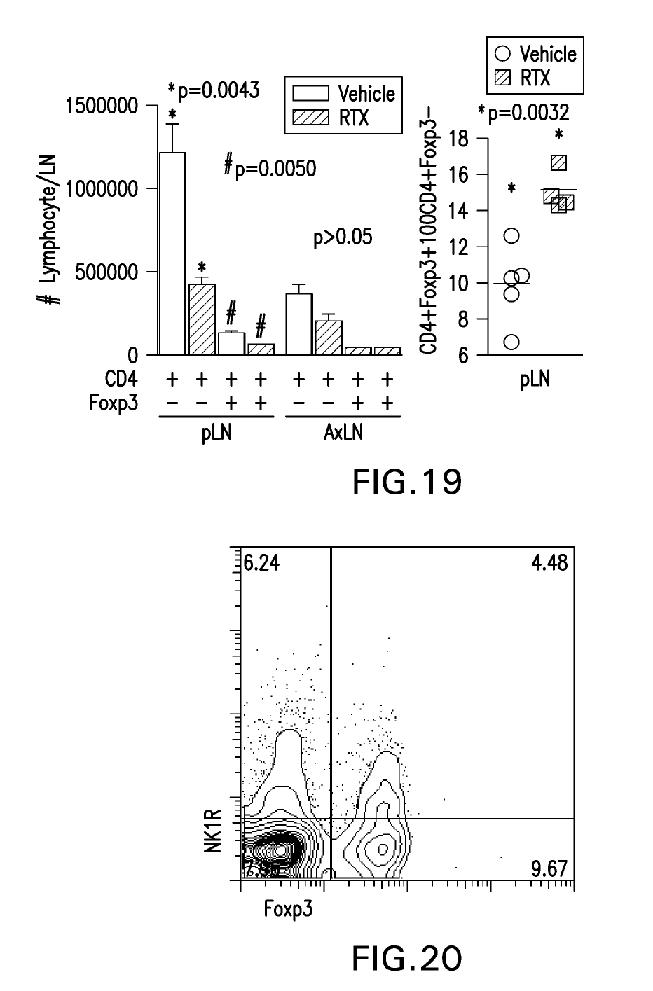

Histological observations are confirmed by quantitative measurements of the effects of TRPV1 agonist (RTX) treatment (FIG. 18). The cellularity of pancreatic lymph nodes (pLN) is dramatically reduced (e.g., about 3-fold; p<0.0001) while the absolute cellularity of axillary nodes (AxLN) from the same animal remain unchanged. Interestingly, this lymphocyte depletion is not random but shows a distinct bias in favor of regulatory T cell subsets. The CD4+ pancreatic node lymphocyte compartment is severely depleted (FIG. 19, left panel). An apparent trend to somewhat lower values in the CD4+ axillary node compartment is not significant. However, when the ratio of Foxp3+ to Foxp3− populations is compared, it is observed that the CD4+ T cell depletion following local T9-T11 TRPV1 agonist (RTX) application is inverted with selective survival of the CD4+Foxp3+ regulatory T cell subset (FIG. 19, right panel). Thus, the direct targeting of lymphoid effector cells by primary sensory afferent neurons is not random, but sophisticated in altering the balance of immunoregulation within a local tissue lesion. Primary afferents may attract lymphocytes to a tissue site, deplete local populations of lymphocytes, and alter the functionality of these cells significantly. The latter may be due to the fact that many regulatory T cells do not express NK1R, the major sP receptors (FIG. 20). See, e.g. Bilkei-Gorzo, A. et al. (2005), supra.

### Local Application of TRPV1Agonist Normalizes Elevated Insulin Resistance.

A core element of Diabetes pathoetiology is progressive hyperinsulinism with consequent elevation of insulin resistance, preventing life threatening hypoglycemia. It is reported that the base regulatory circuit involved in this process maps to the interaction of sP from TRPV1 positive sensory afferent terminals with β-cells, whose insulin secretion sets the activation thresholds of TRPV1 channels with tonic sP release at body temperature.

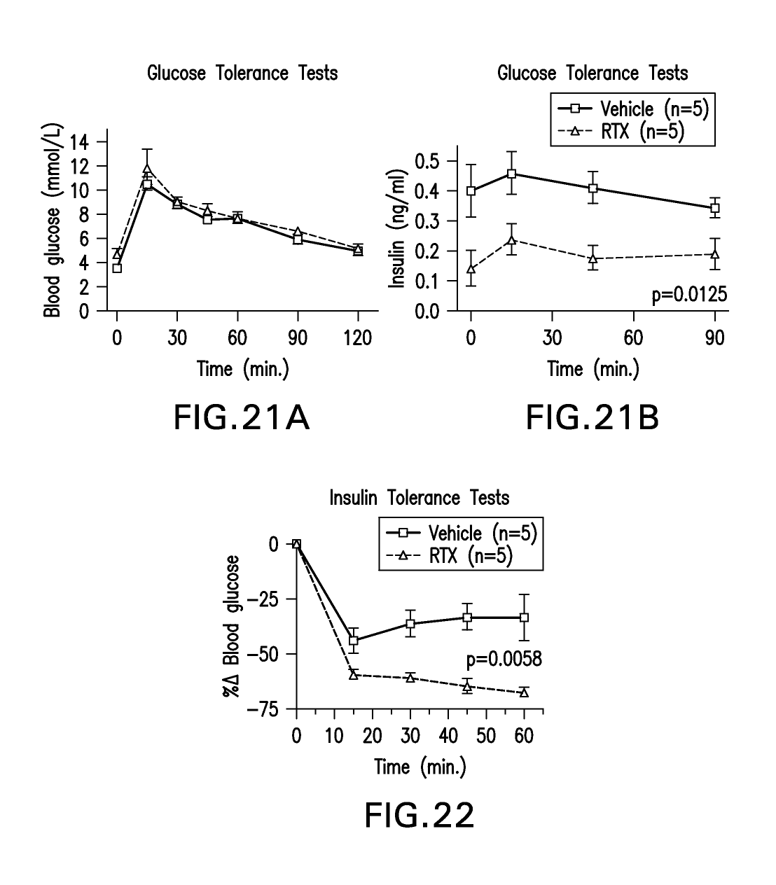

Glucose challenges and insulin tolerance tests are used to determine if local application of TRPV1 agonist affects the pancreatic regulatory circuit in NOD mice. While the response to glucose challenge is nearly identical in RTX-treated versus vehicle-treated control mice (FIG. 21, left panel), there is a major reduction in the high basal blood insulin levels in TRPV1 agonist (RTX) treated mice (FIG. 21, right panel). In addition, the response to glucose challenge in TRPV1 agonist treated mice requires much lower levels of insulin. These observations indicate that local TRPV1 agonist application normalizes hyperinsulinism and abnormal insulin resistance of NOD mice. This conclusion is tested more directly by measuring glucose levels following injection of insulin without prior glucose challenge in TRPV1 agonist treated versus vehicle-treated control mice, although glucagon and hepatic gluconeogenesis may contribute at later time points. These data show improved insulin sensitivity in TRPV1 agonist (RTX) treated versus vehicle-treated control mice (FIG. 22).

### Reversal of Diabetes by Local Application of TRPV1 Agonist.

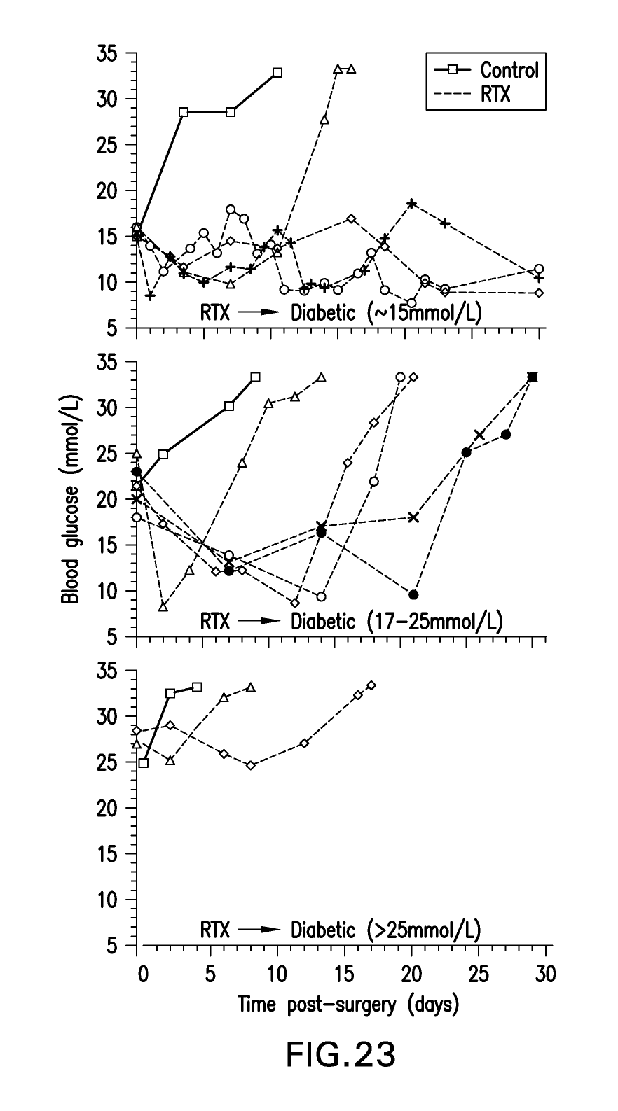

Collectively, these metabolic studies show that local application of TRPV1 agonist (RTX) to pancreas-innervating sensory thoracic wall neurons faithfully reproduces all the effects of intra-arterial pancreas injections with sP without any need for exogenous sP supply. Whether these effects of local TRPV1 agonist (RTX) application will also reverse whole animal diabetes shortly after onset of the disease is important for potential therapeutic strategies in humans. In support, most animals undergoing TRPV1 agonist (RTX) treatment reverted diabetes overnight with non-fasting glucose levels at high-normal, but sustainable, levels (FIG. 23). When results are stratified according to individual glucose levels at the time of presentation of acute diabetes, it is observed that lower glucose levels correlate with the improved success rate and the longevity of TRPV1 agonist (RTX) treatment. At very high initial glucose levels, the TRPV1 agonist (RTX) effect is transient. However, TRPV1 agonist (RTX) mediated reversal of diabetes is improved with respect to response rate and longevity at moderate or borderline glucose levels at the time of onset.

Improved survival rates and diabetes reversal are further observed over two months in animals with 15-17 mmol glucose/L at first presentation (not shown). Importantly, repetition of TRPV1 agonist (RTX) treatment is possible and almost always successful in treating new remissions (not shown). These animals do not receive insulin or any other supportive therapy that might avoid glucose toxicity and promote possible β-cell regeneration at the high normal remission levels of glucose achieved by the treatment.

TRPV1 agonist application to thoracic wall sensory nerve bundles from T8-T12 represents a promising therapeutic alternative to intra-pancreatic sP injection or surgical axotomy. Unlike axotomy, this strategy may be repeated in the same individual to maintain the reversal or avoidance of diabetes progression. It is worth noting that a two month reversal of diabetes in mice may be equivalent to several years in humans, and these time frames with near normal glucose control would be expected from DCCT-EDIC data to have major impact on long term complications. See, e.g. Nathan, D. M. et al., “Intensive diabetes treatment and cardiovascular disease in patients with type 1 diabetes,” N Engl J Med 353:2643-2653 (2005), the entire contents and disclosures of which are hereby incorporated by reference.

While the present invention has been disclosed with references to certain embodiments, numerous modification, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method, comprising the following steps:
(a) identifying an individual having or at risk of developing diabetes, pre-diabetes, or abnormal glucose metabolism; and
(b) stimulating one or more intercostal or subcostal nerves derived from one or more of the following thoracic segments: T8, T9, T10, T11, and T12.

2. The method of claim 1, wherein the individual has one or more of the following symptoms or pathological signs: elevated fasting or non-fasting glucose levels, fasting or non-fasting hyperinsulinemia, glucose intolerance, insulin resistance, dyslipidemia, or hepatic steatosis.

3. The method of claim 1, wherein the individual is a human individual.

4. The method of claim 3, wherein the individual has at least one of the following diseases or conditions: type 1 diabetes (T1D), type 2 diabetes (T2D), type 3 diabetes (T3D), gestational diabetes, type 1.5 diabetes, or latent autoimmune diabetes of the adult (LADA).

5. The method of claim 3, wherein the individual has a body mass index (BMI) within a range of about 25 to about 30.

6. The method of claim 3, wherein the individual has a body mass index (BMI) of about 30 or greater.

7. The method of claim 3, wherein the individual is an individual having pre-diabetes or abnormal glucose regulation or metabolism or at risk of developing diabetes.

8. The method of claim 7, wherein the individual has a fasting or preprandial blood glucose level in a range of about 5.5 mmol per liter to about 7.0 mmol per liter.

9. The method of claim 7, wherein the individual has a blood glucose level in a range of about 7.8 mmol per liter to about 11.1 mmol per liter in an oral glucose tolerance test (OGTT) about two hours after ingesting a 75-gram glucose drink.

10. The method of claim 3, wherein the individual has diabetes.

11. The method of claim 10, wherein the individual has a fasting or preprandial blood glucose level of about 7.0 mmol per liter or greater.

12. The method of claim 10, wherein the individual has a blood glucose level of about 11.1 mmol per liter or greater in an oral glucose tolerance test (OGTT) about two hours after ingesting a 75-gram glucose drink.

13. The method of claim 1, wherein each of the one or more intercostal or subcostal nerves are stimulated unilaterally for each segment.

14. The method of claim 1, wherein the one or more intercostal or subcostal nerves are stimulated at a location distal to their respective dorsal root ganglion (DRG).

15. The method of claim 1, wherein the one or more intercostal or subcostal nerves are stimulated at a location near their respective dorsal root ganglion (DRG).

16. The method of claim 1, wherein the one or more intercostal or subcostal nerves are stimulated at a location distal to the branching point of the dorsal ramus of the spinal nerve.

17. The method of claim 1, wherein the one or more intercostal or subcostal nerves are stimulated by exposure of the one or more intercostal or subcostal nerves to a TRPV1 agonist during step (b).

18. The method of claim 17, wherein the TRPV1 agonist is a capsaicinoid compound or a capsaicin analog.

19. The method of claim 18, wherein the TRPV1 agonist comprises one or more of the following: capsaicin, dihydrocapsaicin, nordihydrocaposaicin, homodihydrocapsaicin, homocapsaicin, or resiniferatoxin (RTX).

20. The method of claim 18, wherein the TRPV1 agonist is capsaicin.

21. The method of claim 1, wherein the one or more intercostal or subcostal nerves are stimulated by exposure of the one or more intercostal or subcostal nerves to a pharmaceutical composition comprising a TRPV1 agonist and a pharmaceutically acceptable carrier.

22. The method of claim 21, wherein the pharmaceutical composition comprises a therapeutically effective amount of a TRPV1 agonist.

23. The method of claim 22, wherein the therapeutically effective amount of a TRPV1 agonist is an amount of the TRPV1 agonist effective to reduce or normalize one or more clinical symptoms or pathological signs of diabetes, pre-diabetes, or abnormal glucose metabolism.

24. The method of claim 22, wherein the therapeutically effective amount of a TRPV1 agonist is an amount of the TRPV1 agonist effective to reduce or normalize one or more of the following clinical symptoms or pathological signs: fasting or non-fasting glucose levels, insulin resistance, glucose intolerance, or fasting or non-fasting hyperinsulinemia.

25. The method of claim 1, further comprising step (c) of making one or more surgical incisions at one or more locations to access the one or more intercostal or subcostal nerves prior to step (b).

26. The method of claim 17, wherein the one or more intercostal or subcostal nerves are exposed to the TRPV1 agonist by local injection of the TRPV1 agonist.

27. A method, comprising the following steps:
(a) identifying an individual having one or more of the following symptoms or pathological signs: elevated fasting or non-fasting glucose levels, fasting or non-fasting hyperinsulinemia, glucose intolerance, insulin resistance, dyslipidemia, or hepatic steatosis; and
(b) stimulating one or more intercostal or subcostal nerves derived from one or more of the following thoracic segments: T8, T9, T10, T11, and T12.

28. A method, comprising the following steps:
(a) identifying an individual having or at risk of developing diabetes, pre-diabetes, or abnormal glucose metabolism; and
(b) administering a composition comprising a TRPV1 agonist to the pancreas of the individual.

29. The method of claim 28, wherein the TRPV1 agonist comprises a capsaicinoid compound or a capsaicin analog.

30. The method of claim 29, wherein the TRPV1 agonist comprises one or more of the following: capsaicin, dihydrocapsaicin, nordihydrocaposaicin, homodihydrocapsaicin, homocapsaicin, or resiniferatoxin (RTX).

31. The method of claim 30, wherein the TRPV1 agonist comprises capsaicin.

32. The method of claim 30, wherein the composition is a pharmaceutical composition comprising a TRPV1 agonist in combination with a pharmaceutically acceptable carrier.

33. The method of claim 28, wherein the TRPV1 agonist is administered by intra-arterial (i.a.) injection into the pancreas of the individual.

34. A method, comprising the following steps:
(a) identifying an individual having or at risk of developing diabetes, pre-diabetes, or abnormal glucose metabolism; and
(b) administering a composition comprising a tachykinin peptide to the pancreas of the individual.

35. The method of claim 33, wherein the tachykinin peptide comprises one or more of the following: substance P (sP), neurokinin A, neurokinin K, neuropeptide gamma, or neurokinin B, or a precursor thereof.

36. The method of claim 34, wherein the tachykinin peptide comprises substance P (sP).

37. The method of claim 33, wherein the composition is a pharmaceutical composition comprising a tachykinin peptide in combination with a pharmaceutically acceptable carrier.

38. The method of claim 33, wherein the tachykinin peptide is administered by intra-arterial (i.a.) injection into the pancreas of the individual.
